(19) United States
(12) Patent Application Publication
(10) Pub. No.: US 2010/0323385 A1
(43) Pub. Date: Dec. 23, 2010

(54) NITROGEN INDEPENDENCE IDENTIFIES A HIGHLY MALIGNANT POPULATION OF TUMOR STEM CELLS

(75) Inventor: Rossella Galli, Milan (IT)

Correspondence Address: ABELMAN, FRAYNE & SCHWAB, 666 THIRD AVENUE, 10TH FLOOR, NEW YORK, NY, 10017, US

(73) Assignee: FONDAZIONE CENTRO SAN RAFFAELE DEL MONTE TABOR, Milano (IT)

(21) Appl. No.: 12/449,607

(22) PCT Filed: Feb. 15, 2008
(86) PCT No.: PCT/EP2008/051860

§ 371 Date: Aug. 14, 2009

Related U.S. Application Data
(60) Provisional application No. 60/902,017, filed on Feb. 16, 2007.

(51) Int. Cl. C12N 5/095; C12Q 1/02; C12N 15/09

(52) U.S. Cl. 435/29; 435/366; 435/368; 435/455

(57) ABSTRACT

The present invention is directed to a method for isolating and establishing Growth Factor-Independent (GF-I) Tumor Stem Cells (TSCs) from tumor biopsies or tumor cell lines consisting in culturing cells in serum-free mitogen-free culture medium. The method discloses cell growth in a culture medium, which does neither comprise serum, nor EGF (Epidermal Growth factor) and FGF-2 (Fibroblast Growth Factor), nor both, nor EGF or FGF-2 derivatives with the same mitogenic characteristics of the parent molecules. According to a preferred embodiment, the method is directed to the isolation of Tumor stem cells (TSCs) from glioblastoma multiforme (GBM) or from other brain tumors or brain tumor cell lines. GF-Independent TSCs can be identified and expanded in vitro providing a homogeneous population of multipotent, self-renewing and highly tumorigenic Growth Factor-Independent TSCs, distinguishable from tumor stem cells derived with other methods, grown in parallel, for the above characteristics. The invention also encompasses therapeutic methods based on Tumor Stem Cells isolated as described.

### FIELD OF THE INVENTION

The present invention is directed to a method for isolating and establishing Growth Factor-Independent (GF-I) Tumor Stem Cells (TSCs) from tumor biopsies or tumor cell lines consisting in culturing cells in serum-free mitogen-free culture medium.

### PRIOR ART

Mitogenic growth factors (GFs) (Perona, 2006) play a critical role in the control of the proliferation and self-renewal of normal somatic stem cells (SCs), including those derived from the central nervous system (CNS) (Reynolds and Weiss, 1992). Intriguingly, the signaling pathways activated by these mitogens are often up regulated in tumors and directly contribute to oncogenesis through the activation of both autocrine and paracrine mechanisms. This phenomenon has been clearly demonstrated for hematopoietic malignancies and it is also involved in the process of gliomagenesis.

In the last ten years, the identification and isolation of putative tumor stem cells (TSCs) has been reported for different types of tumors, including leukemia and breast cancer. Very recently, TSCs have been identified within brain tumors as glioblastoma multiforme (GBM), medulloblastoma and ependymoma (Vescovi et al., 2006). These studies employed the in vitro neurosphere assay (NSA) (Reynolds and Rietze, 2005), which is based on the continuous exposure of cells to epidermal growth factor (EGF) and fibroblast growth factor-2 (FGF-2), in order to progressively enrich the putative stem cells within the original heterogeneous tumor cell population (Galli et al., 2004) (Lee et al., 2006). In addition, GBM TSCs have also been purified by the expression of the surface antigen AC133 (Singh et al., 2004).

Importantly, mitogenic stimulation allowed the isolation and the enrichment of TSCs, which were capable of generating experimental tumors, whose phenotype resembled that of primary human tumors more faithfully than tumors generated from the commonly employed glioma cell lines (Galli et al., 2004) (Lee et al., 2006).

However, in vitro exposure to EGF or FGF-2 is known to promote important functional changes in neural progenitors. Stimulation of adult subventricular zone (SVZ) cells with EGF induces the generation of neurospheres, not only from quiescent type B stem cells, but also from type C transit-amplifying progenitors. This suggests that EGF can alter the normal differentiation program in the SVZ. Similarly, culturing of fate-committed embryonic spinal cord precursors or bipotent astroglial/oligodendroglial O2A progenitors in the presence of FGF2 affects their differentiation competence, by inducing the acquisition of a developmental improper tri-potent cell fate in vitro, or the reversion to a state resembling that of multipotent NSCs, respectively. Thus, GFs appear to critically affect the basic properties of neural progenitors.

The concept of mitogen-induced functional alterations might be applied also to other tumor cells. Since in these cells growth factor stimulation would result in profound modifications of tumor-associated features we have developed a method to culture tumor cells in the absence of mitogenic stimulation. By this methodology long-term cultures of growth factor-independent (GF-I) Tumor Stem Cells have been established.

### SUMMARY OF THE INVENTION

The present invention is directed to a method for isolating and establishing Growth Factor-Independent (GF-I) Tumor Stem Cells (TSCs) from a tumor biopsy or a tumor cell line consisting in culturing cells in serum-free mitogen-free culture medium.

The method discloses growth conditions in a culture medium which does neither comprise serum, nor EGF (Epidermal Growth factor) and FGF-2 (Fibroblast Growth Factor), nor both, nor EGF or FGF-2 derivatives with the same mitogenic characteristics of the parent molecules.

According to a preferred embodiment, the method is directed to the isolation of Tumor stem cells (TSCs) from glioblastoma multiforme (GBM) or from other brain tumors or brain tumor cell lines.

Growth Factor-Independent TSCs can be identified and expanded in vitro providing a homogeneous population of multipotent, self-renewing and highly tumorigenic Growth Factor-Independent TSCs, distinguishable from tumor stem cells derived with other methods, grown in parallel, for the above characteristics. Despite their reduced proliferation in vitro, GF-I TSCs have dramatically increased tumorigenic potential, which is reflected by a pro-invasive molecular signature and by peculiar characteristics. According to a preferred embodiment, said TSC cells are Glioblastoma Multiforme derived stem cells. As confirmed in GBM derived TSCs, the concurrent proliferative and invasive ability of TSCs obtained in mitogen-free condition recalls the phenotype of the highly infiltrative cells of human GBMs. Therefore GF-I TSCs isolated according to the present invention represent an unprecedented and highly reliable tool for the preparation of in vitro and in vivo models by which developing therapies specifically tailored to target the most aggressive cell component of tumors.

### BRIEF DESCRIPTION OF THE DRAWINGS

FIG. 1. Growth factor-independent (GF-I) TSCs are retrieved from established growth factor-dependent (GF-D) GBM TSC lines in the absence of mitogenic stimulation.

(A) Phase-bright microphotographs of neurospheres from GF-D and GF-I TSC cultures show clear differences in clone morphology. Bar: 60 μm. (B) Differences in the growth kinetics were detected between GF-D, GF-I and GF-I/D TSC lines. (C) The growth profile of GF-I TSCs is biphasic, consisting in an early selection phase and a late expansion stage. (D) Serial clonogenic assays show that GF-I TSCs display the same clonal efficiency of GF-D TSCs. (E) Quantitative analysis of the frequency of neurons, astrocytes and oligodendrocytes in both TSC types is summarized. Mean±S.E.M., Student t test, P<0.05, n=3. (F) Immuno-fluorescence (IF) for neuronal (Tuj1, white), glial (GFAP, white) and oligo-dendroglial (GalC, white) markers demonstrates the multipotency of GF-I TSCs. Bar: 60 μm.

FIG. 2. GF-I TSCs are endowed with enhanced tumorigenic ability.

(A) Forty days following intracranial implantation, tumors derived from GF-I TSCs were more extended and invasive than those generated by GF-D TSCs, as shown by MRI (upper panels, sagittal and coronal sections) and by human-specific IF (lower panels, human nuclei, green/light grey). (B) The volumes of GF-I-derived tumors were significantly larger than those of tumors derived from GF-D TSCs. MRI-based volumetric analysis; Student t test, P<0.05; n=10. (C) Kaplan-Meier plot of overall survival of animals transplanted with GF-I and GF-D TSCs. Log-rank test, P<0.0001; n=20. (D) GF-I TSC-derived tumors showed a higher mitotic index than GF-D tumors (40 days post-transplantation, DPT; Student t test, P<0.05, n=3). (E) Mean vessel area was increased in GF-I tumors (Student t test, P<0.001; n=4). (F) Whereas CD147+ vessels (red/diffuse grey) in GF-D tumors were small, regular in shape and uniformly associated to pericytes (NG2, green peripheral staining), blood vessels in GF-I tumors were enlarged and discontinuously covered by NG2+ pericytes. Bar: 60 μm.

FIG. 3. Gene expression profiling and cell morphology indicates that GF-I TSCs are endowed with a highly invasive phenotype. (A) In vitro GF-I TSCs migrated and invaded more efficiently than GF-D TSCs. U87: human astrocytoma cell line as reference for tumor cells; HFNSCs: human fetal neural stem cells as reference for normal neural stem cells. (B) The highly migratory and invasive phenotype of GF-I TSCs correlated with the presence of a peculiar organization of the cytoskeleton, as shown by phalloidin (upper left picture panels) and gelsolin (lower left pictures panels) staining. Ring-like actin bundles: arrow (right panel). Bar in smaller panels: 20 μm. Bar in larger panel: 6 μm.

FIG. 4. EGF-R dependent signaling is hyperactivated in GBM GF-I TSCs.

(A) EGF-R was highly expressed in GBM GF-I TSCs. RT-PCR with EGFR specific primers. Lane 1: GF-D TSCs; lane 2: GF-I TSCs. (B) The enhanced expression of EGF-R in GF-I TSCs was confirmed at the protein level. Western-Blot (upper left panel, anti-EGFR). Increased phosphorylation of the EGF-R (right panels) and strong activation of MAP kinase- and Akt-dependent intracellular cascades was observed mostly in GF-I TSCs (left panel, anti-phosphoERK1 and ERK2 and antiphosphoAkt). Lane 1: GF-D TSCs; lane 2: GF-I TSCs; lane 3: GF-I/D TSCs. (C) Upper panels. Most of the GF-I TSCs were positive for EGF-R, whereas only a subpopulation of GF-D TSCs and GFI/D TSCs was labeled for the same antigen (EGF-R, cytoplasmic green staining; TO-PRO3, TP3, dark blue nuclear staining). Bar: 60 μm. Lower panels. FACS analysis. Left histograms show high-level mean fluorescence intensity (MFI) of EGF-R in GF-I TSCs (blue/dark grey line) as compared to GF-D TSCs (red/light grey line); black line: IgG isotype control. Dot plots on the right show three different cell populations (regions R1, R2 and R3) identified by EGF-R expression and by relative MFI in both types of TSCs. (D) The intense level of EGF-R expression detected in GF-I TSCs by FC correlates with increased receptor density (insets). Intracytoplasmic vacuoles, dashed arrow. (E) EGF-R staining highlighted the presence of structures as filopodia (arrows) and lamellipodia (arrowhead) in GF-I TSCs. Bar in upper panels: 6 μm. Bar in lower panels: 12 μm.

Addendum FIG. 4: (A) Semi-quantitative RT-PCR and Western Blotting for ErbB receptors show that the kinase-inactive ErbB3 is weakly expressed in GF-I TSCs, as compared to GF-D TSCs.

FIG. 5. Mitogen independence correlates with enhanced EGF-R expression and cytoskeleton rearrangements in GBM TSC. (A-C) Short-term analysis of cell viability, apoptosis, and frequency of EGF-R IR cells in GF-D TSCs after mitogen withdrawal. (D-E) A very small percentage of total EGF-R⁺ cells underwent apoptosis after growth factor removal. The majority of EGF-R⁺ cells (cytoplasmic green staining) did not stain for active caspase-3 (nuclear red staining). Confocal microscopy. Nuclear counterstaining: TO-PRO 3, dark blue. Bar: 20 μm. (F) Long-term analysis of growth profile and frequency of EGF-R IR cells in GF-D TSC cultures after mitogen removal. (G) MRI-based volumetric analysis showed that significant differences in tumor volumes were observed when GF-I TSCs grown in the absence of mitogens for seven or more passages were transplanted (30 DPT; Student t test, p<0.05). (H) MRI-based T2-weighted images showed the progressive increase in tumor size according to the sub-culturing passage of GF-I TSCs. (I) Progressive morphological transition from spindle-shaped, elongated cells (arrowheads) to flat, polygonal cells (arrows) (upper panels on the right side) in GF-I TSCs (passage 1-15). EGF-R, green. Nuclear counterstaining: TO-PRO 3 (TP3), dark blue. Bar: 20 μm. Morphological changes were measured by the frequency of cells displaying F-actin ring bundles (dashed arrows) by phalloidin (lower panels, red/light grey).

FIG. 6. EGF-R expression defines a cell hierarchy existing within GBM GF-D TSCs. (A B) Growth curves of EGF-R^{high, low} and ^{neg} GF-D TSCs cultured in the presence (A) or in the absence of mitogens (B). EGF-R^{high}: dark green line with diamond marker; EGF-R^{low}: light green line with square marker; EGF-R^{neg}: black line with triangle marker. (C, D) Volumetric analysis of tumor volumes by MRI at 30 and 50 days after intracranial transplantation indicated that EGF-R expression strongly correlates with the tumorigenic potential of GF-D TSCs (Student t test, P<0.05).

FIG. 7. Long-term proliferating GF-I TSC lines are isolated from primary tumor tissues.

(A) Secondary TSC lines, expanded in vitro from both GF-I and GF-D GBM TSC-derived tumors, displayed similar growth profiles when plated in the presence (black lines) or in the absence (dashed lines) of mitogens; n=3. (B) Expression of EGF-R (green) was detected at similar level and cell frequency in GF-I and GF-D TSC-derived tumors (right panels). Nuclear counterstaining: TO-PRO 3, dark blue. Bar: 15 μm. (C) Phase-bright microphotographs of neurospheres from primary (p)-GF-D and p-GF-I TSC cultures isolated directly from patient's tumor specimens showed clear differences in clone size; n=4. Bar: 120 μm. (D) p-GF-I TSCs expanded in culture more slowly than p-GF-D TSCs but similarly to GF-I TSCs derived from established p-GF-D TSC lines. (E-F) p-GF-I TSCs isolated from a specimen of human anaplastic ganglioglioma (AGG) are more tumorigenic and aggressive than their GF-D counterpart, as shown by MRI 3 months post transplantation (E) and by macroscopic analysis 4 months post-transplantation (F).

FIG. 8. EGF-R positive cells from human primary GBM specimens are more tumorigenic than EGF-R negative cells.

(A) Volumetric analysis of tumor volumes by MRI at 75 days after intracranial transplantation indicated that EGF-R expression strongly correlates with the tumorigenic potential of GBM cells (Student t test, P<0.05). (B) MRI-based T2-weighted images showed that significant differences in tumor volumes were observed when EGF-R positive and negative cells from primary tumor tissues were transplanted (75 DPT; Student t test, p<0.05).

FIG. 9. GF-I TSCs can be isolated also from non-neural tumors.

(A) Breast cancer from pMyt as described in Qiu et al., 2004; (BC)-derived p-GF-D and p-GF-I TSCs have been long term cultured in vitro. Whereas GF-D TSCs display homogeneous morphology, GF-I TSCs give rise to monolayer containing specialized and more differentiated areas. (B) BC p-GF-I TSCs long term proliferate, although more slowly than their GF-D counterpart. (C) Volumetric analysis demonstrates that tumors derived from p-GF-I TSCs are larger than those generated by p-GF-D TSCs.

### DETAILED DESCRIPTION OF THE INVENTION

The present invention relates to a method for establishing Growth Factor-Independent (GF-I) Tumor Stem Cells (TSCs) from biopsies of a tumor or a tumor cell line, consisting in culturing cells in serum-free mitogen-free culture medium. Said culture medium is characterized for not comprising any serum or serum derived component nor any mitogenic factor (Perona, 2006); in particular it does neither comprise EGF (Epidermal Growth factor) nor FGF-2 (Fibroblast Growth Factor-2), nor EGF or FGF-2 derivatives with the same growth and/or mitogenic properties. The term EGF or FGF-2 derivatives refers to peptides or polypeptides with an amino acid sequence homology of at least 90% to the active portion of the molecules and able to trigger the same biological responses in vitro (namely cell proliferation and self-renewal) of the whole molecule.

For the purposes of the present invention, the term tumor encompasses primary tumors, their metastases and secondary tumors; the term tumor cell line comprises a tumor stem cell line (i.e. a growth factor dependent tumor stem cell line).

In order to achieve a homogeneous population of TSCs cultured cells are allowed to grow for at least 10 passages in said serum free mitogen free culture medium. According to a preferred embodiment, growth is preferably continued for a number of passages of at least 10 and usually lower than 20, said final values comprising also intermediate values, such as 11, 12, 13 and so on passages. A preferred composition of the culture medium comprises a buffer system, an osmotic regulator and a hormone mixture. An even more preferred culture medium composition further comprises a protein carrier and a heparin-like compound. The buffer system is preferably Hepes and/or NaHCO₃, the osmotic regulator is a non-reducing sugar such as glucose and the hormone mix composition comprises as active principles the following compounds: insulin, transferrin or its derivatives such as apotransferrin, a salt of selenious acid preferably with an alkaline or alkaline-terrous metal, most preferably Na₂SeO₃.5H₂O (selenite), a progenestinic hormone such as progesterone, a polyammine compound such as putrescine. The protein carrier is preferably serum albumin, preferably bovine, the heparin-like compound is heparin and the basal culture medium is a mix of both DMEM and F12.

TSCs in growth factors or serum independent conditions are characterized by a slower proliferation rate than that observed in Growth Factor (or serum)-dependent conditions. As a consequence, in order to verify that bona fide true TSCs have been amplified, the method further comprises culturing cells in parallel, in growth factor dependent conditions, and obtaining proliferation curves from both dependent and independent growth factor conditions for a period of at least 10 subculturing passages, comparing said curves, wherein a slower proliferation curve is indicative that “bona fide” GF-I TSC have been isolated.

TSCs in growth factors or serum independent conditions are characterized also by a higher invasive capacity than that observed in Growth Factor (or serum)-dependent conditions. As a consequence, in order to verify that bona fide TSCs have been amplified, the method further comprises culturing cells in parallel, in growth factor dependent conditions, and measuring the migration or invasion ability of cells grown in both dependent and independent growth factor conditions and comparing them, wherein a higher invasive capacity is indicative that “bona fide” GF-I TSC have been isolated.

A further indication that “bona fide” GF-I TSC have been isolated comprises the characterization of TSC multipotency, which is carried out by plating cells, after at least 10 passages, preferably 12, even more preferably 15, 20 or 30 all in growth factor independent condition, onto an adhesion substrate such as polyornithine or Matrigel® and allowing cells to differentiate into the specific lineages expected from the tissue of origin of the tumor for at least 2 weeks. Differentiated cultures can be fixed with 4% paraformalheyde and stained with tissue specific antibody markers, i.e. for GBM derived TSCs, with neural-specific antibodies such as Tuj1 for neurons, GFAP for astrocytes and GaIC for oligodendrocytes. The frequency of each cell lineage can be quantified by counting the number of immunoreactive cells for each single lineage and dividing the number for the total cell number as assessed by Hoescht counterstaining.

“Bona Fide” Tumor Stem Cells obtained according to the conditions disclosed in the present invention have been called growth factor-independent (GF-I) TSCs for the purposes of the present invention.

For comparison purposes, it is intended that growth factor dependent (GF-D) conditions refer to cell growth conditions in culture medium comprising at least a mitogen or a growth factor, or serum, or that it comprises at least a mitogen selected from EGF, FGF-2 or both, which is added to the culture medium. The method of the invention is applied to tumors and to tumor cell lines preferably from mammals, even more preferably from humans and rodents. In particular to tumor or tumor cell lines derived from brain, from colon, breast and/or pancreas carcinomas or carcinoma cell lines.

Preferred models for deriving TSCs are brain tumors selected from the group consisting of: anaplastic ganglioglioma (AGG), glioblastoma multiforme (GBM) or medulloblastoma.

The invention therefore refers to bone fide TSCs obtained according to the present invention, from the above tumor specimen or tumor cell lines.

In some embodiments, when the starting material is a brain specimen or even more preferably a glioblastoma multiforme biopsy, the method further comprises sorting and/or selecting for EGF-R positive cell population either before or after growth in serum-free or growth factors-free cell culture medium.

The method of amplification of TSCs may also comprise as an optional step, the transformation with heterologous DNA sequences, either cloned in a vector or not, i.e. encoding a reporter gene. For a heterologous sequence it is intended any nucleic acid sequence which has to be introduced into the TSC in vitro by transfection, elettroporation, microinjection or infection

Tumor Stem Cells (TSCs) obtainable according to the method of the present invention are characterized a) by a slow growth in vitro and enhanced proliferation and invasion in vivo, and b) by a highly malignant phenotype, as assessed by either in vivo or in vitro assays.

Growth factor-independent (GF-I) TSCs can be isolated from established growth factor-dependent (GF-D) TSCs upon mitogen withdrawal and display the cardinal features of true TSCs as a) long term self-renewal, b) multipotency, c) in vivo tumorigenesis. Although GF-I TSCs in vitro proliferate significantly slower than GF-D TSCs, in vivo they give rise to highly aggressive and invasive tumors, which forms and progresses dramatically faster than tumors derived from the GF-D counterpart. This indicates that these GF-I TSCs represent a highly malignant population of TSCs, which is also characterized by a specific pro-invasive molecular signature. For GF-I TSC-derived from a fraction of post-surgery specimens of GBM, this includes the enhanced expression of the receptor for the EGF (EGF-R).

The invention hence relates to a method for isolating Tumor Stem Cells (TSCs) from: a) tumor biopsies, preferably from brain, prostate, and breast tumors, b) from tumor stem cell lines, preferably human glioblastoma TSC lines (such as those described in (Galli et al., 2004), mouse medulloblastoma TSC lines (isolated from the Patched mutant mouse (Goodrich et al., 1997), mouse breast cancer TSC lines (isolated from the PyMT transgenic mouse (Qiu et al., 2004), mouse prostate cancer TSC lines (isolated from the TRAMP transgenic mouse (Gingrich et al., 1996) and c) from serum-dependent traditionally established tumor cell lines as glioma U87 (ATCC HTB-14). Cells are preferably enzymatically or mechanically disaggregated in case of a specimen/biopsy. In the case of brain tumors, the preferred method is based on the use of tryptic enzyme as trypsin and papain. In the case of non-neural tumors and among other proteases, type IV collagenase is the enzyme of choice.

During culture in GF-independent conditions cells may undergo the formation of spheres (i.e. neuro-spheres or mammo-spheres). In these cases the cell culture is continued until primary spheres, neurospheres or mammospheres, develop. The isolation of 1^{st} generation spheres may be followed by disaggregation of said primary spheres and by further subculturing cells, at a density of at least 8×10⁴ cells/cm² in medium without serum and without mitogen or growth factors, as described above until 2^{nd} or further generation spheres develop. TSC are isolated after at least 10 passages in the same culture medium without Growth Factors), preferably 12, more preferably 15 passages, or even more preferably 18-20 passages, depending on the cells type.

In the isolation of GF-I TSCs from brain tissues, the method brings to primary neurospheres, which can be isolated when they reach an average diameter not larger than 300 μm, preferably comprised from 100-300 μm. For culture derived from glioblastoma this usually corresponds to a 7-15 days culture.

Glioblastoma stem cells obtainable according to the present invention show typically a AC133^{neg} phenotype. Until now, the expression of this marker has been shown to be specific of human neural stem cells as well as of human tumor stem cells. In the TSCs of the present invention, the absence of AC133 expression together with EGF-R over-expression defines a typical and novel antigenic profile for glioblastoma stem cells obtained according to the present invention, contrary to the prior art disclosures.

The molecular profile is also typical of the GBM TSC GF-independent cells obtained according to the invention. These TSCs show increased expression of the following genes: EGFR, Wnt5a, Wnt8b; MMP11 12 and 16, N-cadherin, desmin, ATM, TIMP1 and 2, CDK6, CCK4, TGF-beta, FGF8, basigin, TNFR, EPS15, cadherin 13, NT4, 5 and 6, as defined by cancer-specific macroarrays (complete listing in FIG. 3a). GF-I GBM TSCs are in fact characterized by the following surface marker profile: EGFRP^{pos}, AC133^{neg}, Wnt5a^{pos}. However, in total, about 38 upregulated genes have been typically found in GBM TSCs (Database Acc. N°): CDK6 (X66365); KRT11 (M98776); DES (U59167); NIK serine/threonine protein kinase (Y10256); chromatin assembly factor 1 p48 subunit (X74262); tyrosine-protein kinase receptor precursor (D17517); DNA-PK (U35835); ATM (U33841); DNA-repair protein XRCC1 (M36089); XPG (L20046); GADD153 (S40706); RAD1 (L24564); RFC37 (M87339); SOD1 (K00065, X02317); PURA (M96684); UBE2A (M74524); RAD23A (D21235); neurogenic locus notch protein (N) (M99437); Wnt-5A (L20861); Wnt-8B (X91940); EGFR (X00588, K03193); Epidermal growth factor receptor substrate (U07707; Z29064); colon carcinoma kinase 4 precursor (U33635+U40271); TNFR1 and 2 (M32315+M55994); caveolin-1 (Z18951, S49856); cysteine-rich fibroblast growth factor receptor (U28811, U64791); MMP11 (X57766); MMP12 (L23808); MMP16 (D50477); metalloproteinase inhibitor 1 precursor (X03124); tissue inhibitor of metalloproteinases 2 (J05593); EMMPRIN (L20471); ras-related C3 botulinum toxin substrate 2 (M64595; M29871); N-cadherin (M34064, X57548); cadherin 13 (CDH13)-(T-cadherin) (L34058-U59289); FGF8 (U36223); glioblastoma-derived T-cell suppressor factor (M22046, Y00083); NT4 (M86528+S41522). TSCs display a self-renewal ability which is lower, preferably about one half of the proliferation ability of Growth factor Dependent Tumor Stem Cells, as measured for example by a proliferation assay and quantified by generating long-term growth curves (for long term it is intended a culture of at least 10 subculturing passages), whose slope represents an index of self-renewal ability. The slope of the curve reflects the number of long-term proliferating cells and predicts the stem cell frequency. The slope can be calculated by using the formula for a straight line, (y=mx+b), where m is the slope and b is the y-intercept. GF-I TSCs, either from brain tumors or from non-neural cancers, are endowed with a self-renewal capacity lower than that of their growth factor-dependent (GF-D) counterpart, i.e. they display a slope that must be at least ½ that of GF-D TSCs.

Also the invasive ability of GF-D TSCs and GF-I TSCs is different: it is higher for GF-I TSCs obtained according to the present invention, preferably at least about twice that of GF-D TSCs invasive phenotype, as measured for example by volume density measure in migration/invasion assay in vitro, as follows: tumor stem cells are plated onto 6-well Transwell® chambers coated with Matrigel® overnight in DMEM/F12 mitogen-free medium. 7-14 days after plating, cells on the upper side of the filters are mechanically removed, and those migrated onto the lower side are fixed and stained by using the cellular staining DiffQuick®. Migration of cells is evaluated by volume density values acquired by a densitometer scanner and compared for the two populations of TSCs.

The above properties (slower growth rate and higher invasivity) characterize TSCs obtained from different tumors, such as those obtainable from brain and breast. Accordingly, the method provides for the preparation of a homogeneous population of multipotent, self-renewing and highly tumorigenic Growth Factor-Independent TSCs, distinguishable from tumor stem cells derived with other methods, grown in parallel, for the above characteristics.

Despite their reduced proliferation in vitro, GF-I TSCs have dramatically increased tumorigenic potential, which is reflected by a pro-invasive molecular signature and by peculiar morphological characteristics.

Because of their highly malignant phenotype, the isolated cells of the present invention represent a critical tool for the development of both in vivo and in vitro preclinical models for highly aggressive tumors, preferably for glioblastoma models, and for other types of malignancies as well, to be exploited for the identification of novel therapeutics and the generation of diagnostic and prognostic assays for these tumors.

TSCs cells are suitable for the in vitro screening of compounds or libraries with anti-tumorigenic activity wherein one of the following response is measured, as compared to untreated and/or Growth factor dependent stem cells: a) short-term self-renewing capacity, b) long-term self-renewing capacity, c) proliferation, d) differentiation, e) migration and/or invasion ability of TSCs, f) morphological modifications, h) modification of the gene expression profile.

In this context, the stem cells described in the present invention may also be engineered by transformation or transduction of at least one heterologous vectors carrying reporter (i.e. green fluorescent protein, GFP, luciferase, etc.) or therapeutically relevant genes.

Further embodiments of the method, which may comprise further additional steps, are described below and in the claims.

### A. Development of In Vitro Assays Based on TSCs Isolated According to the Present Invention.

The GF-I TSC-specific “memory” of the invasive ability of their tumor of origin, which cannot be retrieved in the human tumor stem cells, in particular in the glioma tumor stem cell lines available so far and dramatically reduced in GF-D TSCs, confers to GF-I TSCs an extraordinary potential. In fact, GF-I TSCs represent a relevant experimental tool a) to identify genes involved in the process of brain tumor initiation, progression and recurrence, b) to generate assays aimed at the validation of the molecular fingerprint of such cells as diagnostic/prognostic markers, and c) as therapeutic targets.

For example GF-I TSCs can be further subcloned by culturing in Growth Factor-free medium to isolate mitogen-independent subpopulations showing additional properties, such as resistance to ionizing radiation/radiotherapy or to chemotherapy, or selected for their ability to extrude the dye Hoechst (Kondo et al. 2004) (side population, SP).

Since in TSC derived from glioblastoma, EGF-R positive cells show an enhanced tumorigenic ability and mitogen independence as shown in the present invention, the frequency of EGF-R positive cells in primary tumor samples together with the frequency of mitogen independent neurospheres generated upon in vitro culturing of EGF-R positive cells gives rise to a powerful biological assay which has both diagnostic and prognostic significance.

Therefore a preferred embodiment of the present invention comprises as a prognostic and/or diagnostic assay, the method of the invention for the isolation and amplification of TS cells from a tumor specimen in GF-Independent condition, comprising, as a further step, the characterization of TSC isolated as described above, for the level of EGF-R expression, wherein a higher EGF-R level correlates with poorer prognosis.

Moreover suitable and specifically tailored therapeutic approaches may be designed on each patients' isolated TSCs (as the method is simple and rather inexpensive) these being directed and targeted, as a consequence, against the most aggressive component of tumors (GBMs, or other proliferating/infiltrating tumor stem cells), which gives rise to minimal residual disease, eventually leading to tumor relapse.

By definition, the requirement of GF-I TSCs to grow under very basic culture conditions in order to proliferate, together with their capacity to grow unlimitedly (over 100 passages in colture), allows the development of efficient biological in vitro assays. Several functional parameters, mostly related to stemness, are directly measured in response to imposed stimuli (e.g. chemical compounds and/or biological drugs).

### GF-I TSC-Based Biological In Vitro Assays for Anti-Tumor Drug Screening.

Many functional properties of stem cells can be employed as parameters for the evaluation of the therapeutic efficacy of biological compounds or drugs. Specific assays are available in the art to measure: a) short-term and long-term self-renewing capacity, b) proliferation, c) differentiation, d) migration/invasion ability of TSCs and e) morphological modifications.

### a) Short-Term and Long-Term Self-Renewing Capacity

This assay can be used to select molecules having anti-tumor activity, in particular drugs affecting the self-renewal properties of TSCs, a relevant feature responsible for the maintenance of the stem cell compartment. a) Short-term self-renewal analysis can be measured on individual clonal spheres which are transferred to 15 ml microfuge tubes containing 1 ml of appropriate medium (1 sphere/tube), where cells are dissociated by pipetting in culture medium. The cell suspension is plated in a clean dish of a 48, 24, or 12 well plates (depending on the number of viable cells) and cells incubated at 37° C. in the humidified chamber. Within one hour of plating, the number of cells obtained by dissociation of each clone under the microscope is counted. A subset of these cells will proliferate giving rise to secondary clones. The cloning efficiency is calculated normalizing the number of secondary clones by the total number of cells in the same well, as assessed by direct observation one hour after dissociation.

### b) Long-Term Self-Renewal Analysis (Population Analysis).

Long-term self-renewal is measured on a cell suspension prepared from spheres, washed with culture medium and seeded at a density of about 8×10³ cells/cm² in culture medium in untreated tissue flasks. The total cell number is calculated at first. At each subculturing passage, dissociated cells are re-plated at the standard cell density; then, the total cell number obtained is calculated. The total cell number is divided by the number of cells originally plated and the amplification rate is multiplied to the value obtained at the previous passage. These values plotted against the time, for at least 10 cell passages, define the growth curves representing each cell line: their slope is a direct measure of the self-renewal capacity of the stem cell population.

c) Anti-proliferation properties can be measured for example after plating TSCs from different tumors onto poly-ornithine-coated 96-well plates (1×10⁴ cells/well). Residual proliferation in the presence of an unknown substance is measured by the methyl-³H-thymidine incorporation assay in a β-scintillation counter and compared to the one measured in untreated cells.
d) Differentiation assay. Differentiation in the presence or absence of unknown substances can be measured by growth of TSCs on adhesive substrates: diluted laminin or Matrigel® solution at the concentration according to the manufacter's instructions ( 1/100 of the stock solution in mitogen-free medium). 4×10⁴ cells/cm² are plated in the appropriate volume of mitogen-free medium alone or containing 20 ng/ml of Leukemia inhibitory factor (LIF).
e) Migration/invasion assays. The activity of anti-invasive molecules is usually measured by plating 2×10⁵ cells onto 6-well Transwell® chambers (Corning Costar, Cambridge, Mass.) coated with Matrigel® overnight in DMEM/F12 mitogen-free medium. 7-14 days after plating, cells on the upper side of the filters are mechanically removed, and cells migrated onto the lower side are fixed and stained by using DiffQuick® (Dade Behring, Ill., USA). Migration of cells is evaluated by volume density values acquired by a densitometer scanner.
f) Quantification of morphological modifications by immunostaining for cytoskeleton markers. F-actin determination was carried out by staining cells with TRITC-labelled phalloidin (1:500; SIGMA, St. Louis, Mo., USA) or with anti-gelsolin (1:50, kind gift from Prof. Marchisio, DIBIT, H. San Raffaele, Milan, Italy). The number of invasive cells is quantified by counting the cells displaying evident ring-like actin bundles in their cytoplasm.

Selection of bioactive compounds is carried out by measuring the biological response of treated TSC and by comparing it to one obtained in untreated cells.

### B. GF-I TSC-Based Uses in Human Therapy and Diagnosis.

GF-I TSCs isolated and amplified according to the present invention may be further treated to block their ability to growth, for example by irradiation, treatment with biological molecular inhibitors, traditional chemotherapeutic agents and used in immunotherapy of oncologic patients, or they are genetically modified. According to a preferred embodiment TSCs are isolated and amplified from brain, breast, colon, prostate cancer specimens, then they are preferably characterized and formulated into compositions suitable for their re-introduction into the patients. In such a way immunotherapy of an oncologic patient is tailored to and specific for the most aggressive component of the tumor.

According to a preferred embodiment, the use of GF-I TSC isolated from glioblastoma is useful in immunotherapy for brain tumors, in particular glioblastoma.

Compositions comprising such inactivated or genetically modified cells in a form suitable for administration to an oncologic patient, are comprised within the above therapeutic embodiment.

According to a further embodiment the present invention relates to a tumor prognostic and/or diagnostic method wherein an efficient isolation of GF-I TSC from patient's tumor tissue specimens is associated to malignant diagnosis and/or poor prognosis.

### EXPERIMENTAL RESULTS

### Materials and Methods

A) GF-I TSC Isolation from Unsorted Tumor Tissue

Human tumor tissues obtained as post-surgery samples or biopsies have been collected in complete, ice-cold growth medium containing antibiotics. The tumor tissue has been separated from the remainder of the material (hemorrhagic and necrotic areas) and processed as soon as possible. A digestion mix has been prepared by weighing out papain, cystein and EDTA. For 50 ml of digestion solution: 47.2 mg of papain, 9 mg of cystein and 9 mg of EDTA. Add 30 ml of EBSS to the tube containing papain/cystein/EDTA. 0.5 ml of 0.1°/oDNAse stock and further 20 ml of EBSS have been added, filter sterilize. Warm up culture medium to 37° C. in a thermostatic water-bath.

### Removal and Collection of Tumor Tissue

Place dish under dissecting microscope at high magnification (25×), dissect out the desired tissue fragment(s) and place tissue in a 35-mm dish containing ice-cold PBS.

### Dissociation of Tumor Tissues and Primary Culture.

The pieces of tissues have been placed into the 15 ml tubes containing the papain mix solution with incubation at 37° C. for 30-60 minutes. At the end of the enzymatic incubation, tissues have been pelleted by centrifugation at 110×g for 10 minutes.

The pellet was added with 3 ml of ovomucoid solution (0.7 mg/ml in culture medium) and triturated with a glass Pasteur pipette, until almost no un-dissociated pieces were left.

Cells have been pelleted and afterward seeded at a density of 8×10³ viable cells/cm² in DMEM/F12 medium containing or not EGF/FGF-2 for GF-D and GF-I TSCs respectively. The growth factor-free, chemically defined DMEM/F12 final medium composition is the following: 2 mM L-glutamine, 0.6% glucose, 9.6 μg/ml putrescin, 6.3 μg/ml progesteron, 5.2 ng/ml sodium selenite, 0.025 mg/ml insulin, 0.1 mg/ml transferrin, and 2 μg/ml heparin. Single cells proliferated to form spherical clusters (primary neurospheres). Primary neurospheres from GBMs developed in culture from 3 to 30 days after plating in the presence/or absence of growth factors used.

To obtain neurospheres from EGF-R positive tumor cells, glioblastoma tumor cells were sorted with FITC-conjugated rabbit anti-EGF-R, clone EGFR.1 (1:100; Chemicon), on a Becton Dickinson (San Jose, Calif.) FACS Vantage SE FACSDiVa equipped with Argon Ion and HeNe lasers. EGF-R positive non-tumor cells as endothelial and hemopoietic cells were excluded from sorting after identification by colabelling with CD34 and CD45/11b, respectively.

GF-I TSCs were also derived from GF-D TSC lines (Galli et al., 2004) (Lee et al., 2006), by culturing GF-D TSCs in the absence of growth factors. After 5-15 days, as a consequence of the selection process in the absence of both adhesion substrates and mitogens, death of EGF-R negative cells and the positive selection of the minority of EGF-R positive cells occurs, thus giving rise to a small number of GF-I neurospheres.

### Propagation of GF-I TSCs in Culture.

As opposed to GF-D TSCs, GF-I TSCs gave rise to very compact and tight neurospheres. In some cases, clones needed to be disaggregated by mechanical dissociation associated with enzymatic digestion. Upon sub-culturing, most of the stem cells survive and generate secondary spheres.

Subculture has been carried out when the spheres reached an average diameter of 100-300 μm. The average subculturing time will require, approximately, 7-15 days for GF-I TSCs.

### Macroarrays Hybridization and Analysis

Atlas™ Human Cancer cDNA expression arrays were purchased from BD Biosciences (cat #7742-1) and used according to the manufacturer's instructions. Total RNA from GF-D TSCs, GF-I TSCs and HFNSCs was extracted using the RNeasy Mini kit (Qiagen, Chatsworth, Calif., USA). cDNA was obtained by using Superscript RNase H⁻ Reverse Transcriptase (Gibco, Rockville, Md., USA). All cDNAs were previously normalized throughout a β-Actin RT-PCR.

### Evaluation of TSC Tumorigenicity In Vivo by Subcutaneous and Orthotopic Implantation

Tumorigenicity was determined by injecting a minimum of 100 up to 2×10⁵ GF-D and GF-I TSCs, for orthotopic transplantation, and a minimum of 1×10⁶ up to 3×10⁶ GF-D and GF-I TSCs, for subcutaneous inoculum, either from primary tumor tissues or by established GF-D TSCs, (Galli et al., 2004). Tumor growth was monitored and quantified by MRI. Mice were sacrificed at different times comprised between 1-10 weeks post-injection, according to the cell line originally injected. Hematoxylin and eosin (H&E) staining and IF were performed on 15 μm-thick cryostat sections. Antibodies/antisera used were: mouse anti-human nuclei (1:100; Chemicon, Temecula, Calif., USA), mouse anti-human EGF-R (1:100; Calbiochem, San Diego, Calif., USA), rabbit anti-Ki67 (1:200; Novocastra, Newcastle upon Tyne, UK), rabbit anti-NG2 (1:300; Chemicon) and rat anti-CD147 (1:600; Serotec, Oxford, UK).

### Fluorescence-Activated Cell Sorting (FACS) Analysis and Cell Sorting

GF-D and GF-I TSCs were incubated with 0.5% bovine serum albumine (BSA) in PBS for 30 minutes, and then stained for 30 minutes using the FITC-conjugated rabbit anti-EGF-R, clone EGFR.1 (1:100; Chemicon). Cells were analyzed and sorted on a Becton Dickinson (San Jose, Calif.) FACS Vantage SE FACSDiVa equipped with Argon Ion and HeNe lasers. Cells were identified and electronically gated on forward and orthogonal light scatter signals. Events representing cells binding anti-EGF-R were identified by their light scatter (FSC and SSC) and fluorescence signatures (FITC).

### Results

Identification and Characterization of Growth Factor-Independent (GF-I) TSCs from Established Growth Factor-Dependent (GF-D) GBM TSC Lines.

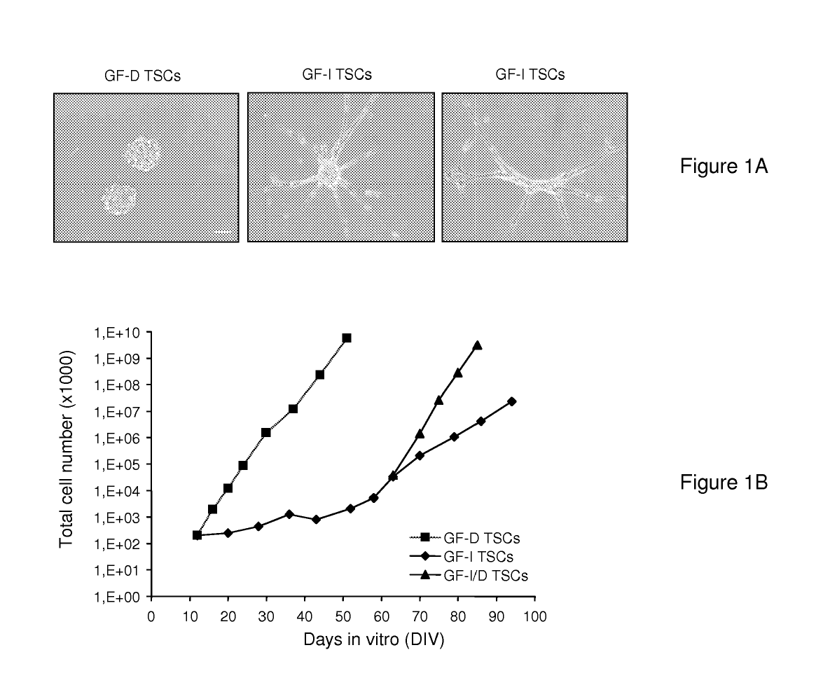

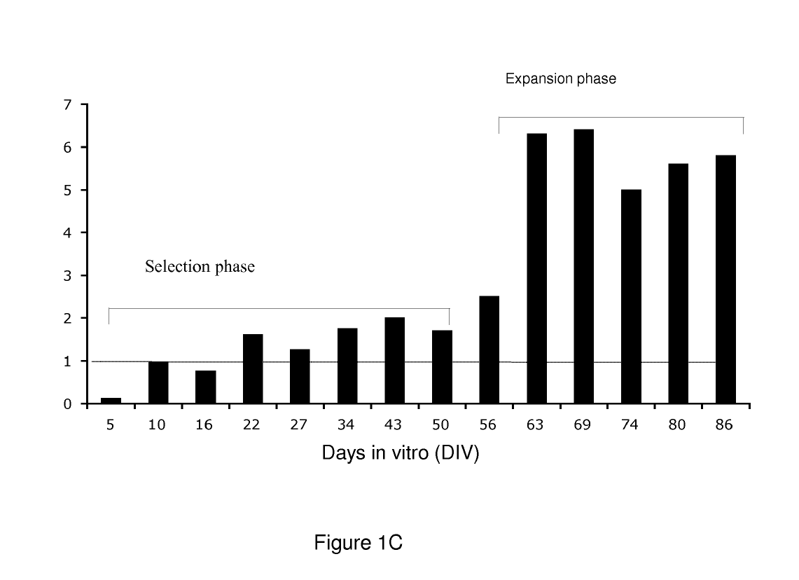

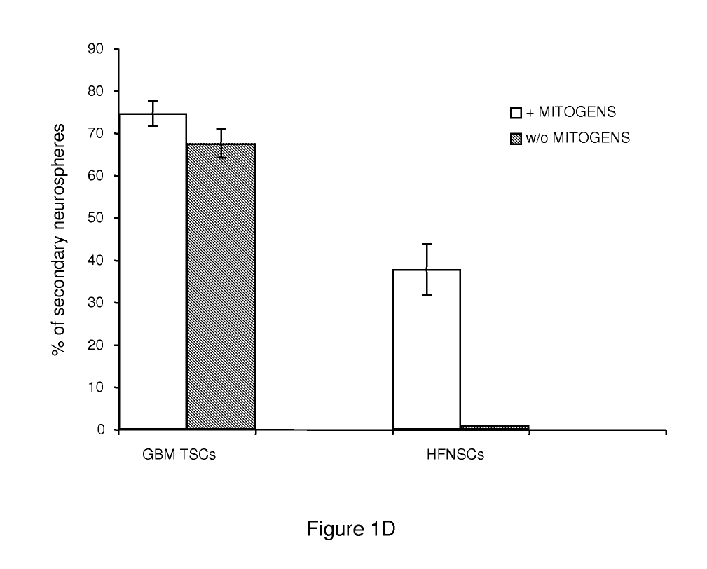

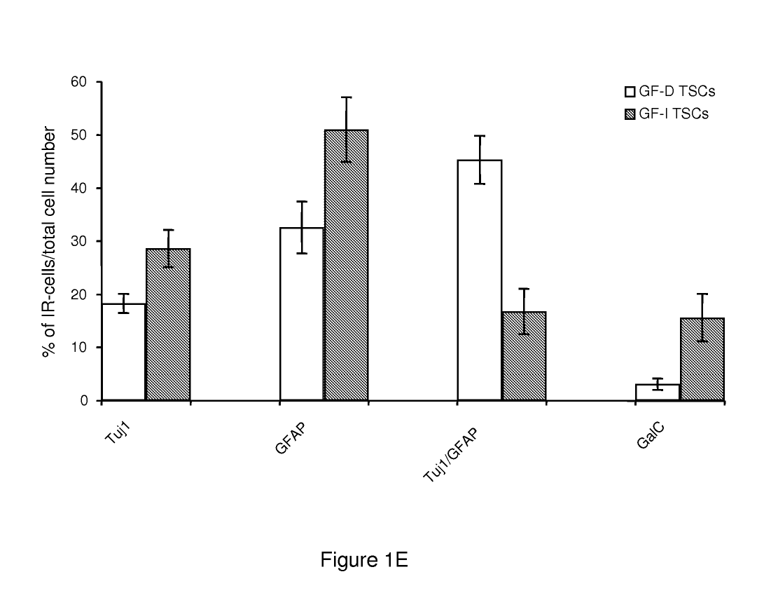

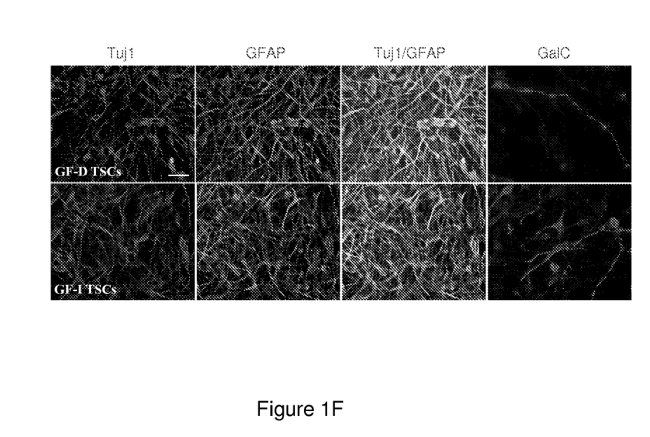

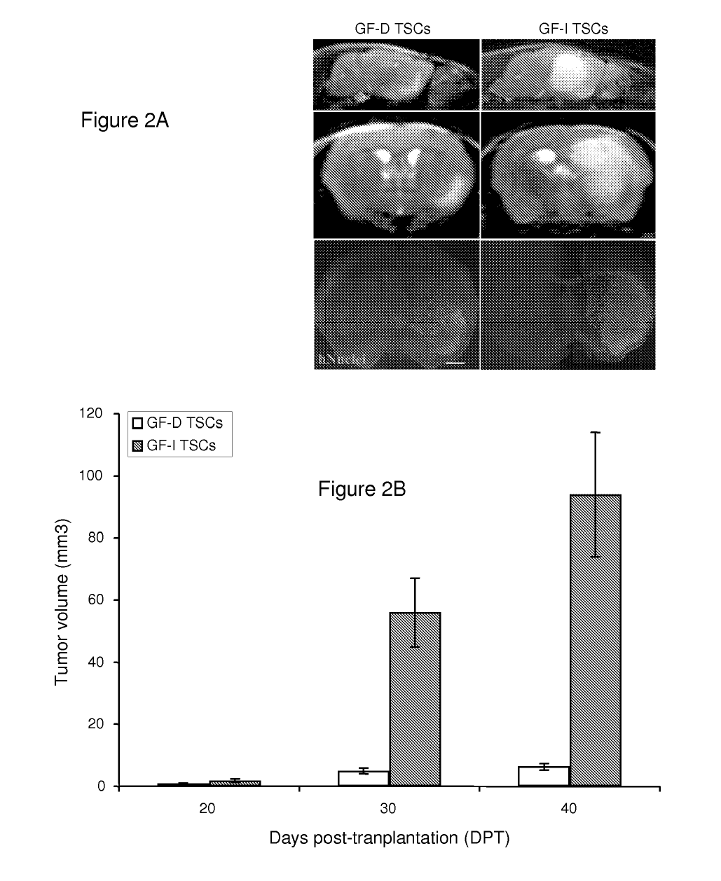

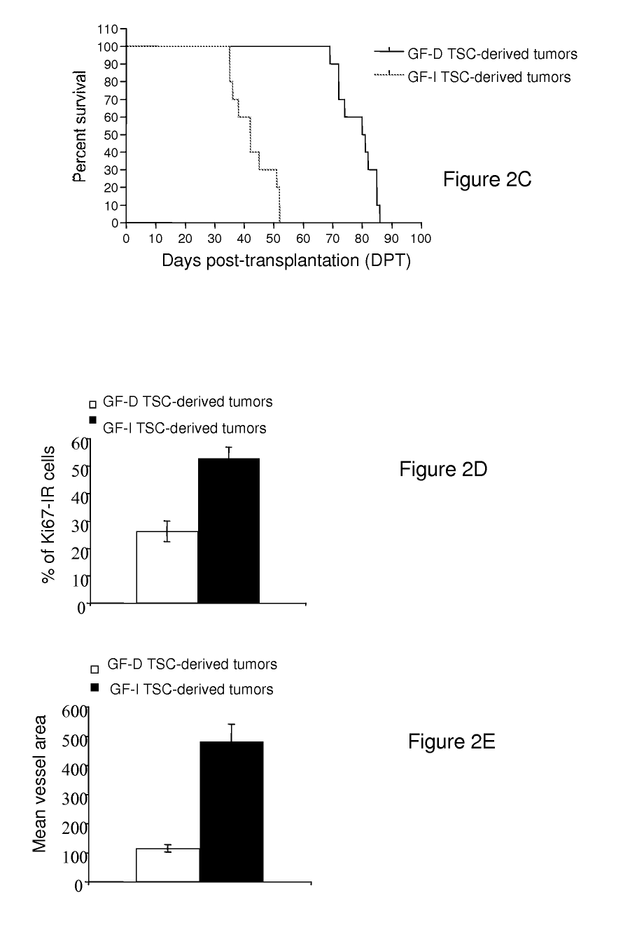

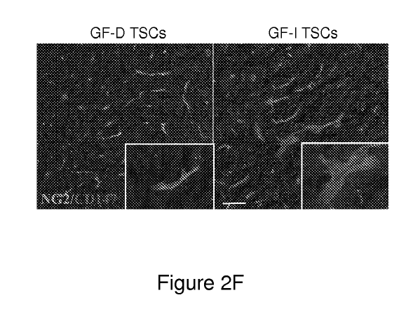

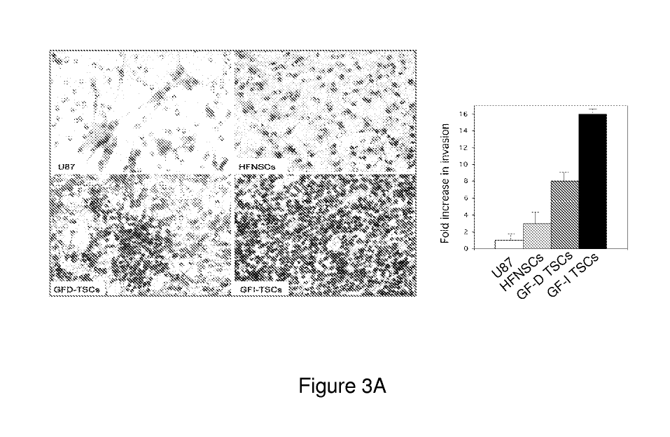

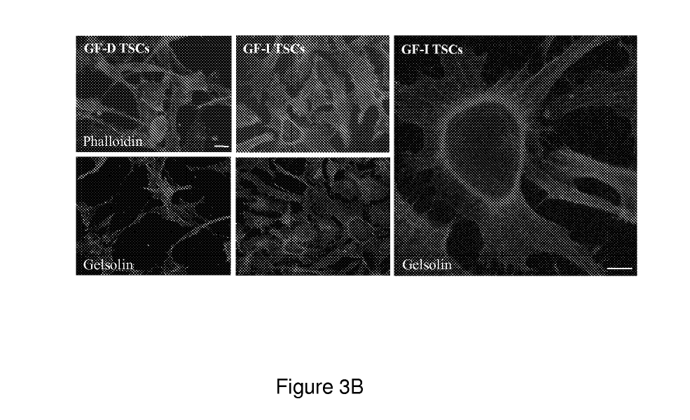

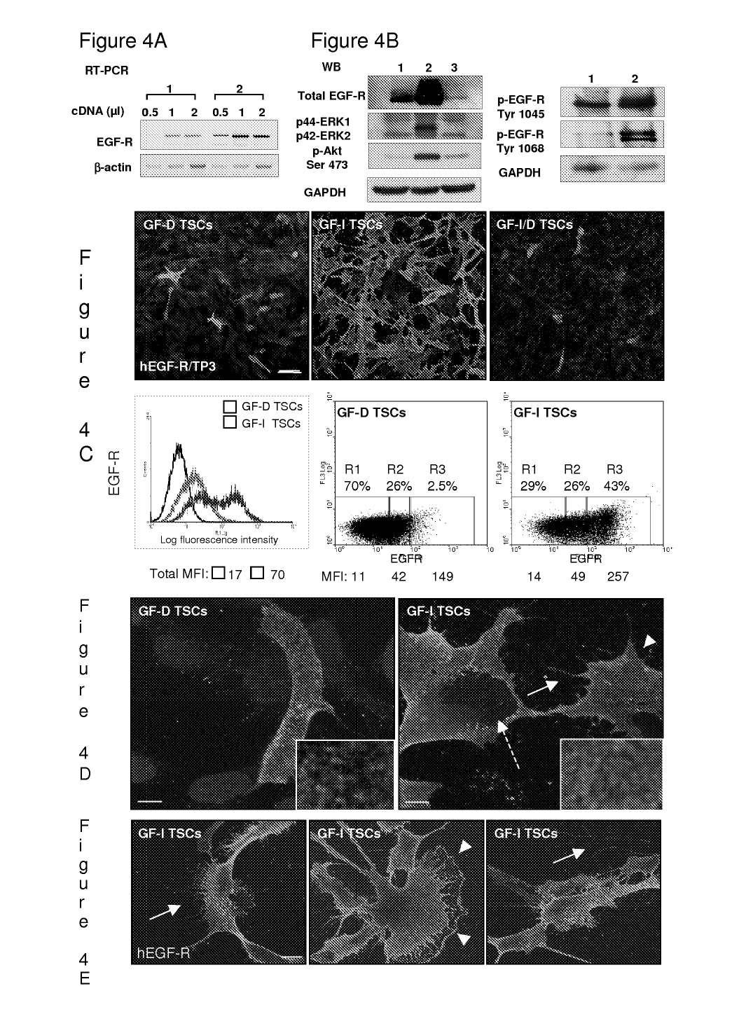

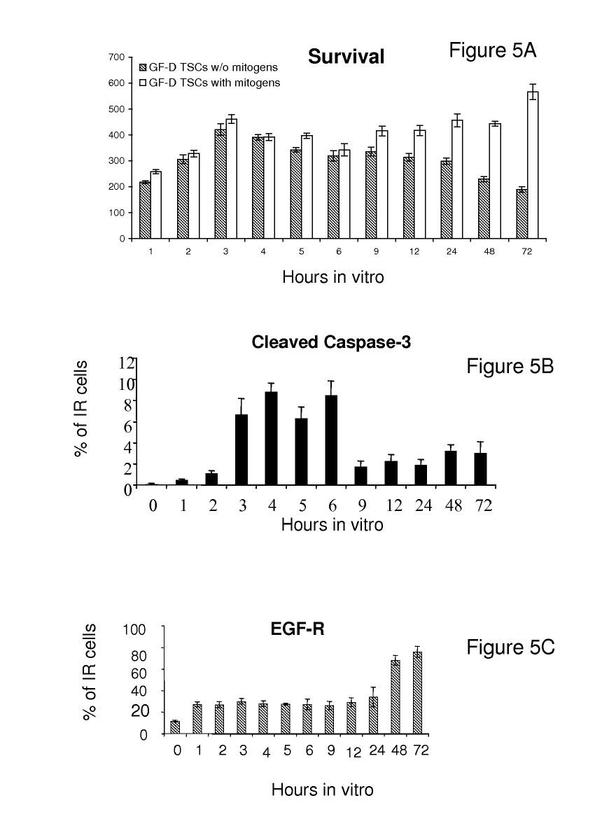

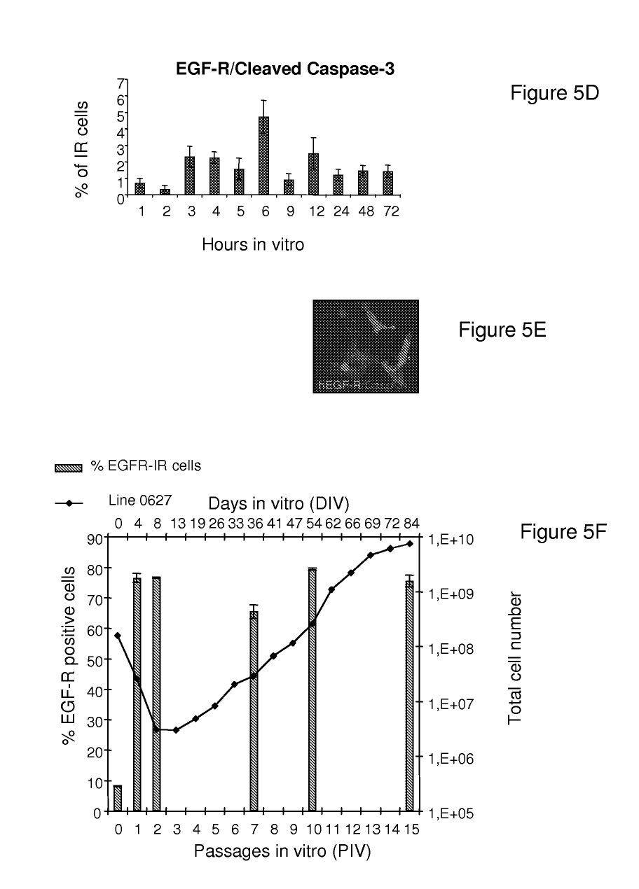

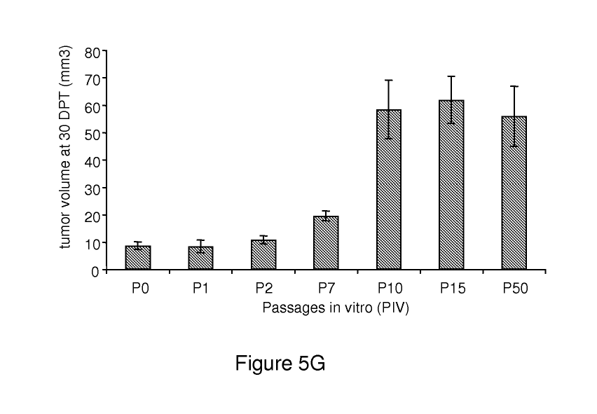

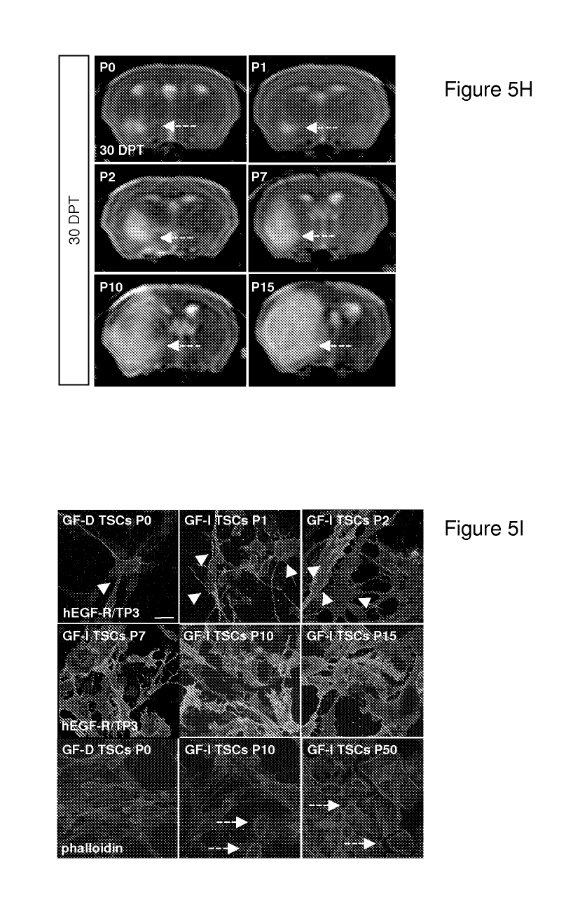

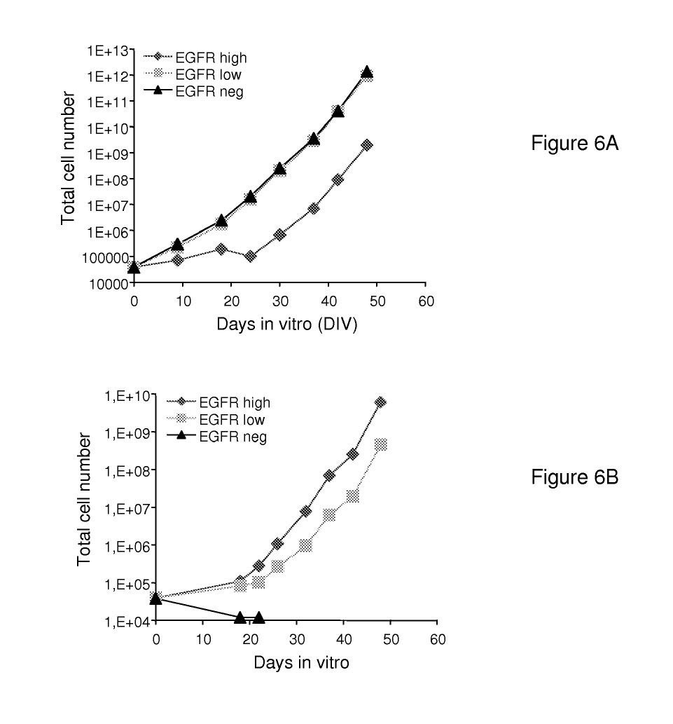

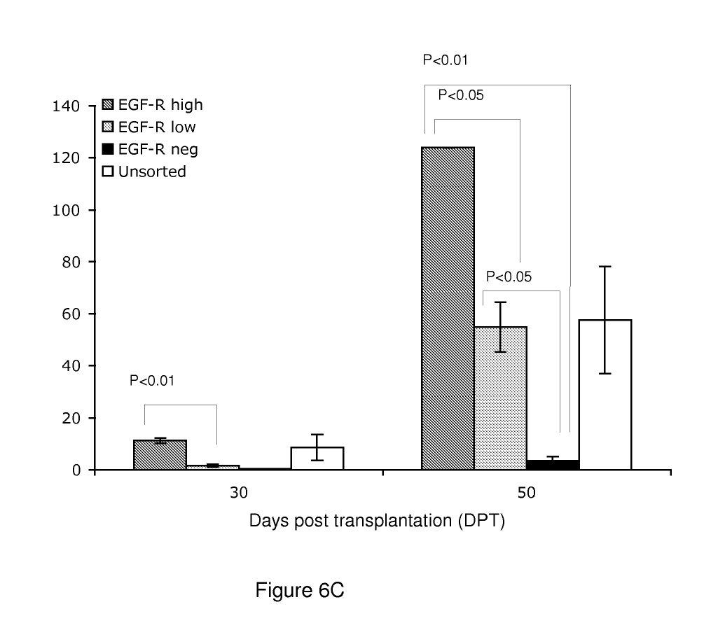

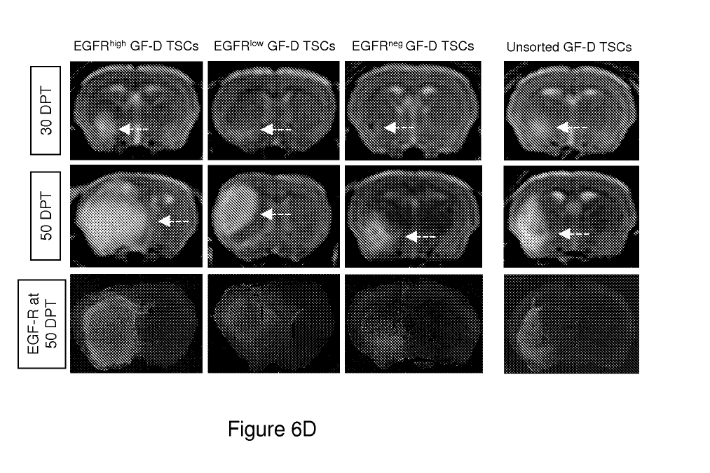

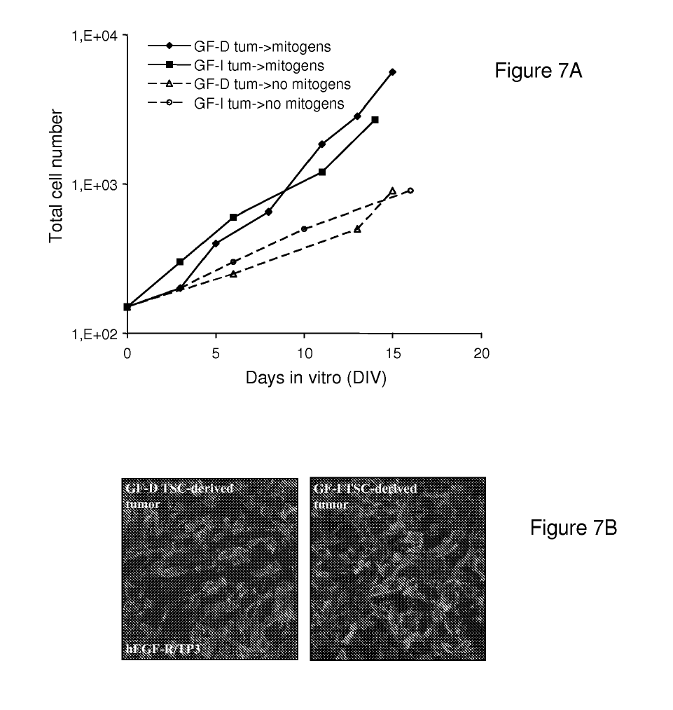

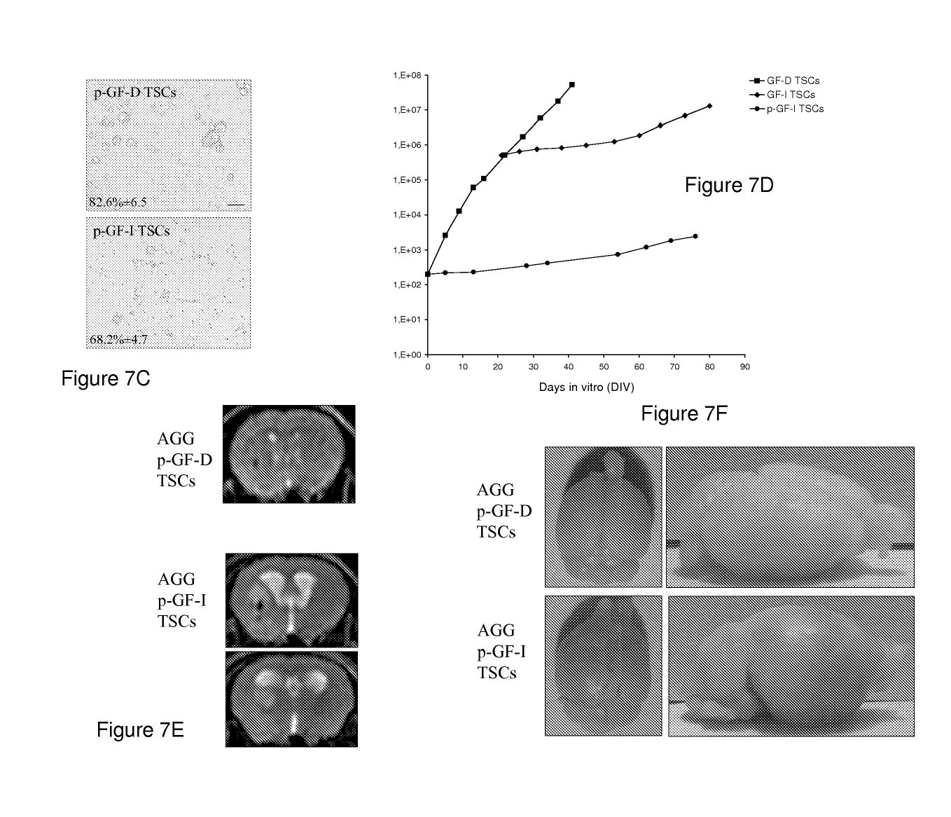

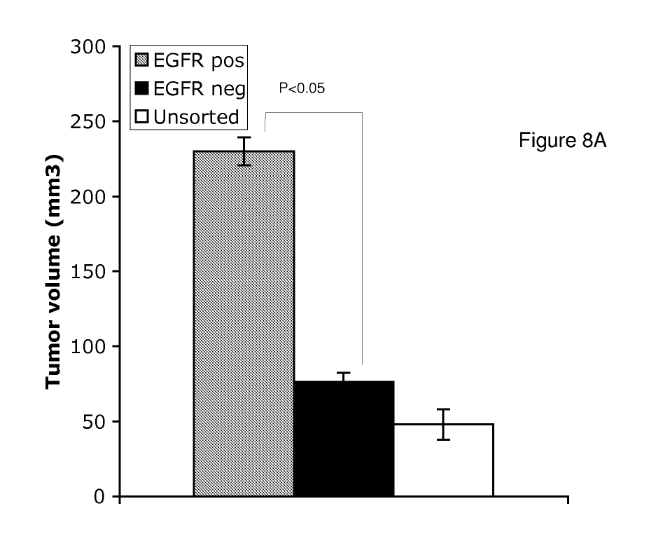

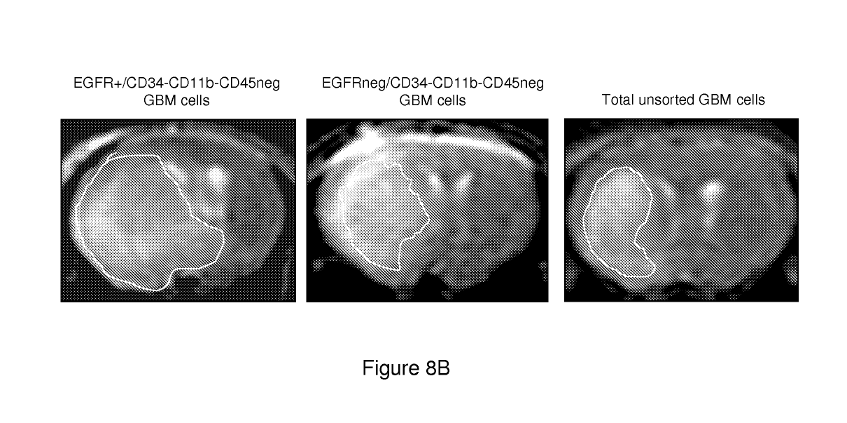

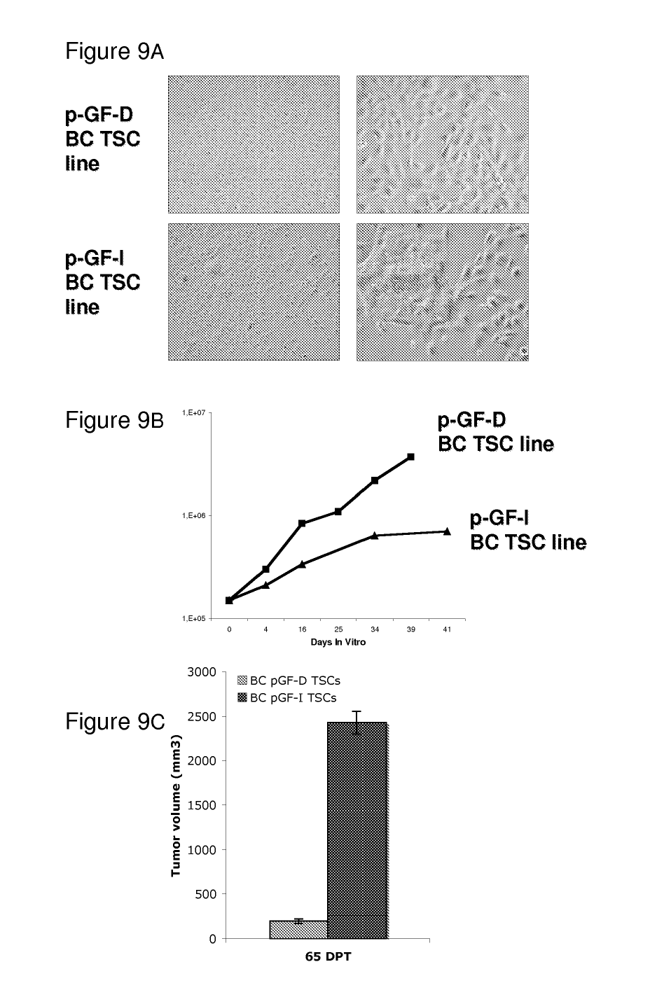

To assess if neurospheres could be generated in the absence of mitogenic stimulation, GF-D TSCs were plated in basal medium devoid of EGF and FGF-2. Withdrawal of the two mitogens from the culture medium induced a high rate of cell death in TSC cultures (not shown) but allowed to a fraction of TSCs to proliferate and to form floating primary neurospheres after 4-10 days in culture. Interestingly, neurospheres from GF-D TSCs displayed conventional morphology (FIG. 1A), whereas neurospheres from GF-I cells were irregularly shaped and characterized by the presence of many process-bearing elongated cells, suggestive of increased cell adhesion (FIG. 1A).

These GF-I cells could be successfully long-term expanded in vitro, proving their extensive self-renewal capacity. Interestingly, the global growth trend of GF-I cells, assessed from the fourth passage onward by population analysis, was strikingly low when compared to that of GF-D TSCs, suggesting the presence of a reduced number of stem cells or changes in their proliferative ability (FIG. 1B). However, when GF-I cells were re-exposed to the same combination of EGF and FGF2, the resulting growth factor-independent/dependent (GF-I/D) TSCs re-acquired the same growth characteristics displayed by GF-D TSCs (FIG. 1B), implying that GF-I TSCs were able to modify their proliferative behavior according to extrinsic stimulation.

Remarkably, the growth kinetics of GF-I cells suggested that progressive positive selection of a cell subpopulation occurred during mitogen-free cell culture (FIG. 1C). Very low amplification rates could be detected at the early stages of culturing following mitogen withdrawal (≦2; passage 1^{st}-5^{th}). However, the expansion rate rapidly increased within few in vitro passages, allowing the establishment of GF-I cell lines (≧2.5; passage 6^{th}-11^{th}).

To analyze the short-term self-renewal ability and the symmetry of division of GF-I cells, we performed sub-cloning assays. The number of secondary neurospheres generated from the dissociation of an individual clone was normalized to the number of originally seeded cells (clonal efficiency). No significant differences could be detected between GF-D cells and GF-I cells in terms of clonal efficiency (FIG. 1D). However, neurospheres generated in GF-I cell cultures were smaller than those derived from GF-D TSCs, suggesting that GF-I TSCs were endowed with reduced proliferative ability, which, in turn, might explain the low rate of expansion of GF-I cells in vitro (FIG. 1B).

As determined by immuno-fluorescence (IF) staining of lineage-specific markers, GF-I cells were able to differentiate into the three neural lineages, i.e. neurons, astrocytes and oligodendrocytes, thus proving their multipotency. Following quantitative analysis, a statistically significant increase in the frequency of each lineage was detected in GF-I cultures (FIGS. 1E and 1F). In agreement with the enhancement of cell differentiation in GF-I cells, a substantial decrease in the number of tumor cells, simultaneously labeled with neuronal and astroglial markers was observed. Since double labeling is an indicator of aberrant stem cell differentiation (Galli et al., 2004; Lee et al., 2006), the absence of mitogenic stimulation improved the proper differentiation of TSCs into single lineage-restricted progeny (FIG. 1F).

As opposed to self-renewal ability and differentiation potential, tumor-associated properties as aberrant karyotype and telomerase activity (Galli et al., 2004) were similar between the two culture conditions.

Therefore, by satisfying the requirements of self-renewal and multipotency in vitro, GF-I cells demonstrated to behave as bona fide stem cells and have been defined as GF-I TSCs.

Orthotopic Xenografts of GF-I TSCs Display Enhanced Growth and Invasive Capability with Respect to GF-D TSCs.

To evaluate if mitogen withdrawal might affect the tumorigenic ability of GBM TSCs, we transplanted GF-I and GF-D TSCs into the brain of nude mice and found that both types of TSCs developed into tumors, closely resembling the invasive phenotype of the human pathology (Galli et al., 2004). Remarkably, tumors generated from GF-I TSCs were much more extended and invaded the brain parenchyma more efficiently than those from GF-D TSCs (n=20), as shown by magnetic resonance imaging (MRI)-based volumetry and serial histological reconstruction by IF of human-specific antigens (FIGS. 2A and 2B). The enhanced tumorigenic ability of GF-I TSCs significantly affected the overall survival of the mice. Indeed, mice injected with GF-I TSCs had a median survival of only 42 days, whereas those injected with GF-D TSCs survived for 80.5 days (FIG. 2C; Kaplan-Meier survival analysis). Interestingly, when GF-I/D TSCs were transplanted orthotopically (n=4), they formed tumors indistinguishable from those generated by GF-D TSCs, in terms of extension, proliferation and angiogenesis (data not shown).

The highly malignant behavior of intracranially transplanted GF-I TSCs was preserved when these cells were injected subcutaneously. GF-I TSC-derived tumors displayed full penetrance and enhanced oncogenicity, as opposed to tumors generated from GF-D TSCs.

Forty days after transplantation, histological analysis of intracranial tumors derived from GF-D and GF-I TSCs showed that they were similar based on hematoxylin and eosin (H&E) staining and the expression of the glial marker GFAP (not shown). However, GF-I TSC-derived tumors proliferated more rapidly than those derived from GF-D TSCs, as shown by quantitative analysis of the proliferation marker Ki67 (FIG. 2D).

When we determined the extent of tumor vascularization by staining for the brain-specific endothelial marker CD147, the total vascular area resulted >3-fold higher in GF-I TSC-derived tumors than in GF-D TSCs, as measured by computer-assisted digital image analysis. Since vessel density did not differ between the two tumor types (not shown), it comes that the mean vessel area was significantly increased in tumors originated from GF-I TSCs (FIG. 2E). In addition, whereas blood vessels of GF-D tumors had regular shape, small diameter, and were mostly covered by NG2⁺ pericytes, those in GF-I TSC-derived tumors were abnormally enlarged and tortuous, with strongly reduced pericyte coverage (FIG. 2F). Thus, GF-I tumor blood vessels were in a highly angiogenic state, while those in GF-D tumors had the features of pre-existing, coopted vessels. Interestingly, the differences in the vascularization of GF-I and GF-D TSC-derived experimental tumors well correlate with the angiogenic behavior of human GBMs, which initially grow by taking advantage of pre-existing vessels and activate neo-angiogenesis only when the tumor reaches a critical size.

The differences in proliferation and angiogenesis might be secondary to the marked difference in size between GF-I and GF-D tumors when analyzed at the same time point. However, comparison of GF-D tumors allowed to grow for 60 days with GF-I tumors grown for 40 days only, which, at these time points of analysis, displayed similar size, confirmed that GF-I TSCs had enhanced ability to promote tumor growth and angiogenesis.

### GF-I TSCs are Defined by a Specific Molecular Signature.

To identify genes differentially expressed between GBM GF-D and GF-I TSCs, and possibly determining the malignant phenotype of GF-I TSCs, we exploited cancer-specific macroarrays. Interestingly, the 38 genes up-regulated by mitogen withdrawal related to central tumor processes such as proliferation, cell adhesion and motility, invasion, and angiogenesis (e.g. Wnt5a, Wnt8b, matrix metalloproteinases (MMP) 11 and 16, N-cadherin). Thus, the growth factor-independence phenotype in vitro can be described by a specific program of gene expression, which is likely responsible for the highly malignant behavior of GF-I TSCs in vivo.

Remarkably, Matrigel® (Mixture of extracellular matrix molecules B&D) invasion assays showed that, even in culture, GF-I TSCs invaded more efficiently than GF-D TSCs (FIG. 3A), indicating that the increased migratory and invasive ability of GF-I TSCs might well represent a cell-autonomous trait.

Since cell motility depends on cytoskeletal modifications and rearrangements, we also stained both TSC types with phalloidin and gelsolin (FIG. 3B). While GF-D TSCs consisted of bipolar, elongated cells, showing poorly organized actin filaments, the majority of GF-I TSCs displayed a flat, spreading, fibroblast-like morphology, with F-actin filaments densely organized in stress fibers and ring-like actin bundles (FIG. 3B), confirming the inherent migratory and invasive nature of these TSCs.

### The Expression of EGF-R and the Activation of Downstream EGF-Dependent Pathways are up Regulated in GF-I TSCs.

To identify the putative mechanisms, accounting for the capacity of GF-I TSCs to proliferate in the absence of mitogenic stimulation and underlying their increased malignant behavior, we analyzed the expression of several soluble factors and of their cognate receptors, involved in tumor cell proliferation, angiogenesis and invasiveness. By RT-PCR, we tested the presence of transcripts for EGF, FGF-2, insulin growth factor-1 (IGF-1), platelet-derived GFs (PDGFs) and vascular endothelial GFs (VEGFs) in both GF-I and GF-D TSCs, and none of the transcripts for these factors was differentially expressed (not shown). Consistently, secretion of EGF, FGF-2, PDGF-AA, and VEGF₁₆₅ by ELISA was observed with no significant quantitative differences between both types of TSCs.

Expression of the cognate receptors for these factors was then measured by RT-PCR. EGF-R was strongly up regulated in GF-I TSCs as compared to GF-D TSCs (FIG. 4A). In agreement with the changes in the mRNA expression, western blotting (WB) of total cellular lysates (FIG. 4B) showed a ten-fold increase in EGF-R protein expression in GF-I TSCs with respect to either GF-D TSCs or GF-I/D TSCs. Importantly, WB for the phosphorylated forms of EGF-R showed that the receptor was strongly phosphorylated in GF-I TSCs at the tyrosine residues 1045 and 1068 (FIG. 4B). Thus, under mitogen-free conditions the EGF-R was not only highly expressed but also hyperactivated. Consistently, very high levels of specific ERK-1/2 and Akt phosphorylation were detected mostly in GF-I TSCs (FIG. 4B). Interestingly, when we assessed the expression of the other members of the ErbB receptor family, we observed that, while ErbB2 was evenly expressed between the two cell populations, ErbB3 was downregulated in GF-I TSCs as compared to GF-D TSCs (addendum to FIG. 4)

Interestingly, IF for human EGF-R on both GF-D and GF-I TSC cultures (FIG. 4C) showed that, whereas the majority of GF-I TSCs were intensely immuno-reactive (IR) for EGF-R, only few GF-D TSCs stained positive (75-85% and 5-11%, in GF-I and GF-D TSCs, respectively; Student t test, P<0.0001, n=7). As expected, GF-I/D TSCs displayed a number of EGF-R⁺ cells similar to GF-D TSCs (6.0%±0.4, n=3), suggesting that EGF-R might not be constitutively active in most of the GF-I TSCs (FIG. 4C).

When we analyzed GF-D and GF-I TSCs for EGF-R expression by flow cytometry (FC), we identified three distinct subpopulations within each TSC types. Whereas both GF-D and GF-I TSCs contained similar figures of EGF-R^{low} cells (region R2), the frequency of EGF-R^{high} (region R3) and EGF-R^{neg} (region R1) cells dramatically varied between GF-D and GF-I TSCs, with the latter displaying the highest proportion of intensely labeled cells and the lowest of negative cells (FIG. 4C). In addition, the level of EGF-R expression in each of the different GF-I TSCs subpopulations, shown as mean fluorescence intensity (MFI), was higher than that observed in the corresponding fractions in GF-D TSCs.

FACS analysis for the expression of the putative GBM TSC marker AC133 (Singh, 2003) (Singh et al., 2004) showed that a large proportion (60%) of GF-D TSCs was AC133⁺. Some of these AC133⁺ GF-D TSCs (15%) were also EGF-R⁺. Interestingly, the highly tumorigenic GF-I TSCs were uniformly AC133^{neg}, suggesting that AC133 expression is not a pre-requisite for stemness in the absence of mitogenic stimulation.

Notably, the intense labeling of GF-I TSCs for EGF-R shown by FC (FIG. 4C) well correlated with a corresponding dramatic increase in the density of the receptor on the cell surface, as compared to low-expressing EGF-R⁺ GF-D TSCs (insets in FIG. 4D).

The increased expression the EGF-R on the surface of GF-I TSC reinforced the notion that these cells might be endowed with a motile phenotype. Indeed, by EGF-R staining, the presence of pro-invasive structures such as filopodia and lamellipodia (FIG. 4E), together with malignant cytological features such as intracytoplasmic lumens (FIG. 4D), could be observed in most of the GF-I TSCs (FIG. 4E), confirming their highly invasive nature.

### Mitogen Withdrawal Selects for EGF-R+TSCs, While Eliminating EGF-R^{neg} Cells.

The restricted expression of the EGF-R in a fraction of GF-D TSCs (FIG. 4C) suggested that this subset of EGF-R⁺ cells might indeed represent the stem cells, which, upon mitogen removal, give rise to the GF-I TSCs. To prove this concept, we analyzed the effect of mitogen withdrawal on GF-D TSC survival within a short time window. MTT survival assay showed that GF-D TSCs plated either in the presence or in the absence of mitogens did not differ in terms of total cell viability within the first 4 hours of culture (FIG. 5A). However, a relevant decrease in cell survival started to be observed in GF-D TSCs 5 hours after mitogen removal, and it reached a nadir 72 hours later. Within the same time frame, a wave of cell apoptosis, measured as the frequency of cleaved caspase-3 IR cells, peaked between 3 and 6 hours and continued up to 72 hours after plating (FIG. 5B). Interestingly, the frequency of EGF-R⁺ cells increased from the basal 11% up to 30% as early as one hour after mitogen removal, remained constant for the following 24 hours and, after additional 24 hours, reached a plateau, attesting at the value of 75±3%, characteristic of established GF-I TSCs (FIG. 5C). Intriguingly, very few EGF-R⁺ cells (less than 6% of the total EGF-R⁺ cells) co-stained with cleaved caspase-3, indicating that the TSCs, undergoing cell death after growth factor withdrawal, were those negative for the EGF-R expression (FIGS. 5D and 5E).

To confirm that EGF-R expression in EGF-R⁺ cells supported the progressive enrichment of GF-I TSCs at the expense of EGF-R^{neg} cells, we extended our study up to 15 sub-culturing passages (corresponding to 84 days in vitro) in the absence of mitogens. At every passage, we measured both the global growth trend and the frequency of EGF-R immuno-reactive cells (FIG. 5F). Long-term population analysis pointed out that the same decrease in total cell number by cell death observed in the first 72 hours of selection continued to be detected throughout the first three passages under mitogen-free conditions (selection phase; FIG. 5F and not shown). In agreement with the short-term analysis, at the first passage of selection (i.e. after 4 days in vitro), the number of EGF-R IR cells already increased from basal levels (8.2±0.2% of EGF-R IR cells) up to 76.6±1.5% of the total residual viable cells and remained stable throughout further sub-culturing (FIG. 5F).

However, when we tested the tumorigenic potential of GF-I TSCs at different sub-culturing passages (FIGS. 5G and 5H), a statistically significant difference in the volume of tumors could be detected only when GF-I TSCs from the 7^{th} passage onward were transplanted, although the frequency of EGF-R IR cells was already at plateau at the first passage of selection (FIG. 5F).

Interestingly, the morphology of GF-I TSCs changed gradually throughout sub-culturing (FIG. 5I), from the typical spindle-shaped of GF-D cultures (passages 0 through 6) to the flat and polygonal appearance characteristic of GF-I TSCs (passage 7 onward) (FIGS. 3B and 4E). Indeed, these morphological modifications, quantified based on the development of F-actin ring bundles within the cells (FIG. 5I), strictly correlated with the tumorigenic behavior of GF-I TSCs (frequency of TSCs displaying F-actin ring staining: undetectable at passage 0; 10.2%±0.98 at passage 10; 26.4%±1.24 at passage 50; mean±S.E.M., Student t test, P<0.005). Thus, the aggressive in vivo behavior of long-term established GF-I TSCs might not only depend on the overall EGF-R expression per se, but also on relevant modifications in their morphology, which are indicative of the phenotype of migrating and invasive cells and might be required for GF-I TSCs to become fully malignant (FIG. 5I). Of note, GF-I TSCs at 10^{th} passage onward were able to generate tumors with an average size comparable to those derived from the implantation of GF-I TSCs cultured for more than 50 passages (FIG. 5H), indicating that the mitogen-independent phenotype requires 10-15 passages in the absence of mitogens to be fully revealed.

To determine whether mitogen independence represents a general property of primary GBMs or a feature of GBMs characterized by genetic modifications of the EGF-R, we analyzed a series of GBM TSC lines for the presence of EGF-R gene mutations and/or gene amplification. By RT-PCR, we assessed the presence of the most common form of mutant EGF-R, which harbours an in-frame deletion mutation of exons 2 to 7 spanning the extracellular ligand-binding domain. By using primers flanking exons 2 to 7, two discrete amplicons distinguishing the expression of EGFRvIII (236 bps) from the larger EGFR wild type transcript (1037 bps) could be identified. Co-expression of the transcript of the mutant and the wild-type receptor could be identified in two out of 10 distinct TSC lines (20%), and in their corresponding tissue of origin. Interestingly, the frequency of EGFRvIII positive TSC lines coincided with the percentage of patient cases in which the variant is normally detected (20-30%). However, mitogen independence did not appear to correlate with the presence of the mutant EGFRvIII, since GF-I TSCs could be generated also from TSC lines expressing only the wild-type EGFR.

EGF-R Expression Identifies Three Distinct GBM TSC Subpopulations with Different Growth Factor Requirement and Tumorigenic Potential.

In order to formally demonstrate that the EGF-R⁺ TSC population represents the most aggressive TSC component, able to give rise to GF-I TSCs when challenged by mitogen removal, we set out to purify EGF-R^{high, low} and ^{neg} cells from GF-D TSCs by fluorescence-activated cell sorting (FACS). The purity of each cell fraction was 96-98% (not shown). All cell fractions, obtained by the same gates defined for regions R1, R2 and R3 (FIG. 4C), were tested for long-term self-renewal in vitro as well as for tumorigenic potential in vivo.

When cultured in the presence of GFs, purified EGF-R^{high, low} and ^{neg} GF-D TSCs resumed the typical growth behavior of GF-D TSCs, although a longer period of in vitro adaptation was required for EGF-R^{high} TSCs to proliferate extensively, suggesting that events as receptor down-regulation and morphological changes might be necessary for EGF-R^{high} cells to interact with the mitogen-containing in vitro milieu (FIG. 6A). In contrast, only EGF-R^{high} and EGF-R^{low} GF-D TSCs were able to self-renew extensively when cultured without GFs (FIG. 6B). In fact, plating of EGF-R^{neg} TSCs in the absence of mitogens resulted in a progressive decline in their proliferative capacity, indicating that they might indeed represent the TSCs undergoing progressive negative selection upon mitogen abrogation (FIG. 6B). When induced to differentiate, all the three cell fractions, previously grown in the presence or in the absence of GFs, maintained their multipotency and gave rise to similar percentages of neurons, astrocytes and oligodendrocytes (data not shown).

When the same three cell fractions were transplanted intracranially into nude mice immediately after cell sorting, 30 days following transplantation tumors could be detected in animals receiving EGF-R^{high} GF-D TSC, whereas no sign of tumor development was observed in mice transplanted with equal numbers of EGF-R^{low} and ^{neg} GF-D TSCs (FIGS. 6C and 6D). However, 50 days after transplantation, tumor lesions started to become detectable by MRI also in animals receiving EGF-R^{low} and ^{neg} GF-D TSCs, although their volume was significantly smaller than those of tumors derived from EGF-R^{high} GF-D TSCs. Tumors developing from EGF-R^{high} GF-D TSCs were significantly larger than those from EGF-R^{low} TSCs, which were more extended than those from EGF-R^{neg} TSCs.

Interestingly, tumors derived from the implant of 2×10⁵ unsorted GF-D TSCs (comprising 1×10⁴ EGF-R^{high}, 5×10⁴ EGF-R^{low} and 1.4×10⁵ EGF-R^{neg} TSCs) were smaller than those generated from purified EGF-R^{high} cells, but similar in size to those developing from a double amount of purified EGF-R^{low} cells, suggesting that additional factors, such as paracrine signals, produced by the three different subsets of GF-D TSCs when injected as a whole, might actively contribute to GBM tumorigenesis (FIG. 6C). Finally, in line with the progressive selection experiments, none of the tumors derived from the implantation of purified EGF-R^{high} GF-D TSCs reached the typical size of the lesions generated by GF-I TSCs at the same time points (FIGS. 2B and 5G), confirming that not only an increase in EGF-R expression but also the morphological and molecular changes as those documented in long-term cultured GF-I TSCs (FIG. 3B and FIG. 4E) are required to enhance the extent of GBM TSC tumorigenicity.

EGF-R Positive Cells from Human Primary GBM Specimens are More Tumorigenic than EGF-R Negative Cells.

EGF-R⁺ and ^{neg} tumor cells were isolated from patient's tumor specimens by FACS. To avoid co-purification of tumor cells with putative EGF-R⁺ non-tumor cells, both CD34⁺ endothelial and CD45⁺/CD11b⁺ hemopoietic cells were excluded from the sorting. EGF-R⁺/CD34/CD45/CD11b^{neg} and EGF-R^{neg}/CD34/CD45/CD11b^{neg} tumor cells were: a) cultured in the presence or absence of mitogens; b) directly transplanted into the brain of nude mice without any in vitro manipulation.

Interestingly, primary neurosphere formation was observed when EGF-R⁺ GBM cells were grown either in the presence or in the absence of mitogenic stimulation (6.15%±1.6 and 2.15%±1.1, respectively). Conversely, plating of EGF-R^{neg} GBM cells under both conditions resulted in the generation of very limited numbers of neurospheres, only when cells were exposed to mitogens (0.36%±0.02). Most significantly, when both EGF-R⁺ and ^{neg} GBM cells were intracranially implanted in immunodeficient mice, a striking difference in tumor formation was observed. In fact, when measured 10-12 weeks following transplantation by MRI, EGF-R⁺ GBM cell-derived tumors were three-fold larger than those generated from EGF-R^{neg} cells, thus suggesting that EGF-R expression specifically identifies the most aggressive stem cell component also in primary GBMs (FIG. 8). Mitogen-independent TSCs are constitutively present within experimental and GBM tumors

After the demonstration that GF-I TSCs could be retrieved in GF-D TSC lines, we asked whether these TSCs could be identified and isolated also from fresh tumor tissues, thus ruling out the possibility that GF-I TSCs might develop as a mere consequence of long-term culturing in vitro.

We started by analyzing either GF-D or GF-I TSC-derived experimental tumors for the presence of GF-I TSCs. Forty days after orthotopic tumor implantation, we dissected and enzymatically digested both types of tumor tissues and cultured the cells in the presence (Galli et al., 2004) or absence of EGF and FGF2. In the presence of GFs, secondary TSC lines could be established not only from GF-D TSC-derived tumors, as previously reported (Galli et al., 2004), but also from GF-I TSC-derived tumors. Most interestingly, both tumors contained TSCs capable of proliferating and long-term expanding in the absence of mitogens, thus proving that GF-I TSCs are an inherent component of experimental GBMs in vivo. All the secondary TSCs displayed overlapping growth profiles under both in vitro settings, indicating that GF-D and GF-I TSC-derived tumors were similar in terms of stem cell composition and behavior (FIG. 7A). In agreement with the well-established significance of EGF-R expression in human GBMs (Behin et al., 2003), IF for human EGF-R on tissue sections from GF-D and GF-I-derived tumors indicated that the receptor was present in all the tumor cells (FIG. 7B). Notably, EGF-R was never expressed in tumors generated by the implantation of non invasive U87 cell line (not shown), thus confirming that GF-I and GF-D TSC-derived tumors uniquely and faithfully resemble the features of the human disease in vivo (Galli et al., 2004) (Lee et al., 2006) and that EGF-R might be one of the plausible mediator of the phenotypic/genotypic association between primary and experimental TSC-derived tumors.

In order to verify if the GF-I TSC population might also represent an intrinsic and essential component of human GBMs, we grew equal numbers of freshly dissociated cells from patient's tumor specimens in the presence or in the absence of GFs. Ten to 20 days after plating, similar number of neurospheres could be detected in GF-stimulated cultures (primary, p-GF-D TSCs) and under mitogen-free conditions (p-GF-I TSCs) (FIG. 7C). Consistent with what observed for GF-I TSCs from GF-D TSC lines, clones from p-GF-I cultures were smaller than those from p-GF-D TSCs (FIG. 7C). Remarkably, p-GF-I TSCs, obtained directly from the tumor tissue, proliferated and extensively self-renewed in culture, maintaining the same slow in vitro expansion, observed for the GF-I TSCs, which were later isolated from the established p-GF-D TSCs (FIG. 7D).

Most importantly, when transplanted intracranially, p-GF-I TSCs formed tumors, which resulted more aggressive and extended than those from p-GF-D TSCs, and similar to those generated from the GF-I TSCs later derived from established p-GF-D TSCs (not shown).

To assess whether other subtypes of brain tumors do contain mitogen-independent TSCs, we cultured equal numbers of freshly dissociated cells from patient's tumor specimens of anaplastic ganglioglioma (AGG) in the presence or in the absence of GFs. As previously shown for GBMs, AGG p-GF-I TSCs could be long-term expanded in vitro in the absence of mitogens, giving rise to self-renewing multipotent GF-I TSC lines. Most importantly, following intracranial implantation, AGG p-GF-I TSCs were endowed with dramatically enhanced tumorigenic capacity (FIG. 7), thus confirming previous findings on GBMs. Finally, in order to investigate whether the phenomenon of mitogen independence can be extended to comprise also non-neural tumors, thus hinting to the possibility that mitogen independence might be a feature of many different TSCs, we subjected tumor cells obtained from the enzymatic dissociation of mouse breast cancer (BC), derived from the MMTV-PyMT mouse model (i.e. transgenic mice which express the mouse polyomavirus middle-T antigen under the control of the mouse mammary tumor virus long terminal repeat, Qiu et al. 2004) to our in vitro assay (FIG. 9). Interestingly, BC primary (p-) TSCs could be isolated and expanded in vitro either in the presence (ref) or in the absence of mitogens, giving rise to floating cluster of cells known as mammospheres. However, few days following in vitro subculturing, TSCs started growing as adherent layers of cells under both culture conditions. However, the cell layer produced by BC p-GF-I TSCs was more heterogenous and specialized than that generated by BC p-GF-D TSCs. As shown for GBM TSCs, BC p-GF-I TSCs were characterized by a reduced proliferative rate as compared to BC p-GF-D TSCs. Most importantly, when orthotopically implanted in the lower mammary glands of FVB mice, BC p-GF-I TSCs gave rise to highly proliferative and aggressive tumors, which were 20-times larger than those generated by BC p-GF-D TSCs. Therefore, mitogen independence is not limited to tumors if neural origin, but can be observed also in other neoplasias.

Isolation and Long-Term Culturing of Bona Fide GBM TSCs in the Absence of GFs Allow the Selective Enrichment of Stem Cells (SCs) with Enhanced Tumorigenic Potential.

In order to enter into an active proliferative status, non-tumor normal cells, including stem cells, strictly require mitogenic growth signals, as GFs, extra-cellular matrix components and cell-to-cell adhesion molecules. In particular, neural stem cells (NSCs) have been identified and isolated only as the result of the exposure to specific mitogens as EGF and FGF-2. However, continuous mitogenic stimulation in vitro has been shown to significantly alter the basic physiology of the exposed cells, by inducing phenotypic reversion.

As opposed to normal cells, tumor cells demonstrate strongly reduced growth factor-dependence and self-sufficiency in growth stimuli. In particular, the establishment of EGF-, FGF-2-, PDGF-, and VEGF-dependent autocrine/paracrine loops and over-expression of their receptors, as EGF-R/erbB and PDGF-R, has been specifically implicated in the growth and progression of high-grade gliomas, also under experimental settings. Since growth factor-induced cellular reprogramming in GBM TSCs has been shown to lead to a decrease or even a loss of tumor-associated properties, as tumor antigenicity and stem cell-specific gene expression, the identification of the TSC population, self-supporting in mitogen stimuli, in brain tumors might coincide with the isolation of the most authentic stem cell, the only able to retain relevant tumor-related features.

In the present study, we describe a novel long-term self-renewing population of TSCs, obtained from both GF-D TSCs and post-surgery specimens of human tumors, by in vitro culture in the absence of mitogens. The resulting GF-I TSCs, although displaying in vitro the same characteristics of their GF-D counterpart, as self-renewal and multipotency, demonstrate in vivo an extraordinary tumorigenic ability, which appears to be sustained by increased cell proliferation and angiogenesis and, in this specific case of TSCs isolated from GBMs, correlates also with EGF-R over-expression. Contextually, we propose that the in vitro propagation of GBM TSCs in the presence of mitogens, although supporting robust in vitro proliferation and subsequent cell expansion, might be detrimental on overall TSC features. In fact, continuous exposure to GFs leads to a strong decrease in the stem cell frequency, as indicated by the small fraction of highly malignant EGF-R^{high} TSCs retrieved in mitogen-stimulated cultures, and to functional alterations in TSCs, as a reduction in their invasive and migratory potential.

### The Highly Malignant Tumorigenic Behavior of GF-I TSCs is Regulated Cell-Autonomously.

The peculiar in vivo behavior of GF-I TSCs suggests that these cells are intrinsically different from GF-D TSCs. In fact, in spite of their very low rate of proliferation in vitro, GF-I TSCs completely changed their growth behavior upon transplantation, demonstrating reduced latency in tumor generation and increased malignancy. This enhanced tumorigenic ability is dependent on the inherent pro-invasive and migratory phenotype of GF-I TSCs. Specific genes might be deregulated upon acquisition of self-sufficiency following mitogen withdrawal. By exploiting macroarrays for small-scale transcriptional profiling, we identified a possible “mitogen independence” program, characteristics of GBM TSCs when deprived of growth stimuli. The program included not only genes controlling cell division but also genes involved in the positive regulation of cell migration and invasion as intermediate filament proteins and metalloproteinases. Thus, the enhanced expression of genes, relevant for tumor dispersal, cell proliferation and angiogenesis, in established GF-I TSCs might predispose and prime the cells to interact with the in vivo microenvironment more proficiently than GF-D TSCs, upon transplantation.

It has been proposed that the occurrence of proliferative and invasive capacity within the same tumor cell might play a fundamental role in tumor dissemination. Accordingly, the identification of Growth Factor-Independent TSCs, which retain both stem-ness (i.e. proliferation and self-renewal) and high mobility features, usually mutually exclusive within the same cell, might have a significant impact in the comprehension of the mechanisms underlying malignant evolution of tumors and in particular of brain tumors and gliomas. At the same time, our findings underline how mitogen stimulation, by compelling TSCs to proliferate and self-renew while decreasing their motility, might modify therapeutically relevant traits in these cells, as the overall capacity for invasion and brain dispersal. Mitogen-independent TSCs are retrieved in primary tumors.

Here we report that GF-I TSC lines are identified and isolated not only from established GF-D TSCs but also from patient's tumor specimens (p-GF-I TSCs), indicating that GF-I TSCs do not arise as a consequence of in vitro progressive selection of aggressive sub-clones, which might have accumulated mutations leading to growth factor-independence. Rather, GF-I TSCs represent a cell population constitutively retrievable within patient's tumors, corresponding to the EGF-R positive fraction of tumor cells in the case of GBMs, and indicating that molecules specifically expressed by GF-I TSCs can be exploited for immunotherapy protocols under autologous settings.

### REFERENCES

Behin, A., Hoang-Xuan, K., Carpentier, A. F., and Delattre, J. Y. (2003). Primary brain tumours in adults. Lancet 361, 323-331.
Galli, R., Binda, E., Orfanelli, U., Cipelletti, B., Gritti, A., De Vitis, S., Fiocco, R., Foroni, C., Dimeco, F., and Vescovi, A. (2004). Isolation and Characterization of Tumorigenic, Stem-like Neural Precursors from Human Glioblastoma. Cancer Res 64, 7011-7021.
Gingrich, J. R., Barrios, R. J., Morton, R. A., Boyce, B. F., DeMayo, F. J., Finegold, M. J., Angelopoulou, R., Rosen, J. M., and Greenberg, N. M. (1996). Metastatic prostate cancer in a transgenic mouse. Cancer Res 56, 4096-4102.
Goodrich, L. V., Milenkovic, L., Higgins, K. M., and Scott, M. P. (1997). Altered neural cell fates and medulloblastoma in mouse patched mutants. Science 277, 1109-1113.
Kondo, T., Setoguchi, T., and Taga, T. (2004). Persistence of a small subpopulation of cancer stem-like cells in the C6 glioma cell line. Proc Natl Acad Sci USA 101, 781-786.
Lee, J., Kotliarova, S., Kotliarov, Y., Li, A., Su, Q., Donin, N. M., Pastorino, S., Purow, B. W., Christopher, N., Zhang, W., et al. (2006). Tumor stem cells derived from glioblastomas cultured in bFGF and EGF more closely mirror the phenotype and genotype of primary tumors than do serum-cultured cell lines. Cancer Cell 9, 391-403.
Perona, R. (2006). Cell signalling: growth factors and tyrosine kinase receptors. Clin Transl Oncol 8, 77-82.
Qiu, T. H., Chandramouli, G. V., Hunter, K. W., Alkharouf, N. W., Green, J. E., and Liu, E. T. (2004). Global expression profiling identifies signatures of tumor virulence in MMTV-PyMT-transgenic mice: correlation to human disease. Cancer Res 64, 5973-5981.
Reynolds, B. A., and Rietze, R. L. (2005). Neural stem cells and neurospheres—re-evaluating the relationship. Nat Methods 2, 333-336.
Reynolds, B. A., and Weiss, S. (1992). Generation of neurons and astrocytes from isolated cells of the adult mammalian central nervous system.[comment]. Science 255, 1707-1710.
Singh, C. I., Terasaki M, Bonn V E, Hawkins C, Squire J, Dirks P B. (2003). Identification of a cancer stem cell in human brain tumors. Cancer Research 63, 5821-5828.
Singh, S. K., Hawkins, C., Clarke, I. D., Squire, J. A., Bayani, J., Hide, T., Henkelman, R. M., Cusimano, M. D., and Dirks, P. B. (2004). Identification of human brain tumour initiating cells. Nature 432, 396-401.
Vescovi, A. L., Galli, R., and Reynolds, B. A. (2006). Brain tumour stem cells. Nat Rev Cancer 6, 425-436.

1. A method for isolating and establishing Growth Factor-Independent (GF-I) Tumor Stem Cells (TSCs) from a tumor biopsy or a tumor cell line consisting essentially in culturing isolated cells in serum-free mitogen-free culture medium.

2. The method according to claim 1 wherein said culture medium does neither comprise EGF (Epidermal Growth factor) nor FGF-2 (Fibroblast Growth Factor), nor EGF or FGF-2 derivatives.

3. The method according to claim 2 wherein said culturing is carried out for at least 10 cell passages in said serum free mitogen free culture medium.

4. The method according to claim 3 wherein said culture medium is a mix of DMEM and F12, further comprising at least: a buffer system, an osmotic regulator, a hormone mixture, a protein carrier or

5.-7. (canceled)

8. The method according to claim 3 further comprising culturing cells in growth-factor independent conditions for at least 10 culturing passages.

9. The method according to claim 5 further comprising culturing isolated cells from a tumor biopsy or from a tumor cell line, in parallel, in growth-factor dependent (GF-D) conditions, obtaining proliferation curves from both dependent and independent growth factor conditions for at least 10 culturing passages, comparing said curves, wherein a slower proliferation curve in GF-I is indicative that “bona fide” GF-I TSC have been isolated.

10.-12. (canceled)

13. The method according to claim 1 wherein said tumor or tumor cell line is human.

14. The method according to claim 13 wherein said tumor or tumor cell line is from brain, colon, breast and/or pancreas.

15. The method according to claim 14 wherein said brain tumor is a human anaplastic ganglioglioma (AGG), a glioblastoma multiforme (GBM) or a medulloblastoma.

16. (canceled)

17. The method according to claim 8 further comprising sorting and/or selecting for a EGF-R positive cell population either before or after growth in serum-free or mitogen-free cell culture medium.

18. The method according to claim 1 further comprising a step of transformation with heterologous DNA sequences.

19. A Growth Factor Independent Tumor Stem Cell (GF-I TSC), prepared according to the method of claim 8.

20. A Growth Factor Independent Glioblastoma Tumor Stem Cell (GF-I GBM TSC) obtainable according to the method of claim 9 and characterized by the following surface markers immunologic profile: EGF-R^{pos} (positivity) AC133^{neg} (negativity) Wnt5a^{pos} (positivity).

21. A method for screening compounds with anti-tumorigenic activity comprising the method for isolating and establishing Growth Factor-Independent (GF-I) Tumor Stern Cells (TSCs) from a tumor biopsy or a tumor cell line according to claim 1 and further comprising measuring at least one among a cell response selected from the group consisting of a) short-term self-renewing capacity, b) long-term self-renewing capacity, c) proliferation, d) differentiation, e) migration and/or invasion ability of TSCs, f) morphological modifications, and g) gene expression profile, and comparing said cell response to the response of untreated cells or of the same cells grown in Growth Factor Dependent conditions.

22.-39. (canceled)

40. The method according to claim 8 further comprising adding suitable excipients and/or diluents for preparing an immunogenic composition comprising TSCs.
